(19) United States
(12) Patent Application Publication
(10) Pub. No.: US 2026/0248851 A1
(43) Pub. Date: Aug. 27, 2026

(54) GRANZYME EXPRESSING T CELLS AND METHODS OF USE

(71) Applicants: Jill SLANSKY, Denver, CO (US); Zachary HAY, Denver, CO (US); The Regents of the University of Colorado, A Body Corporate, Denver, CO (US)

(72) Inventors: Jill SLANSKY, Denver, CO (US); Zachary HAY, Denver, CO (US)

(21) Appl. No.: 18/854,097

(22) PCT Filed: Apr. 6, 2023
(86) PCT No.: PCT/US2023/065463

§ 371 Date: Oct. 4, 2024

Related U.S. Application Data
(60) Provisional application No. 63/327,875, filed on Apr. 6, 2022.
(60) Provisional application No. 63/339,104, filed on May 6, 2022.

(51) Int. Cl. A61K 35/17; A61K 35/12; A61K 40/11; A61K 40/31; A61K 40/42; A61P 35/00; C07K 14/705; C12N 5/0783; C12N 5/0789; C12N 9/64

(52) U.S. Cl. CPC A61K 35/17; A61K 40/11; A61K 40/31; A61K 40/42; A61P 35/00; C07K 14/70503; C12N 5/0636; C12N 5/0647; C12N 9/6467; C12Y 304/21078; C12Y 304/21079; A61K 2035/124; C12N 2510/00; C12N 2830/34

(57) ABSTRACT

The disclosure describes immune cells that express granzyme protease(s), methods of producing and methods of using the immune cells for the treatment of cancer.

### CROSS-REFERENCE TO RELATED APPLICATIONS

The present application claims priority to, and the benefit of U.S. Provisional Patent Application No. 63/327,875, filed Apr. 6, 2022 and U.S. Provisional Patent Application No. 63/339,104, filed May 6, 2022, each of which is incorporated herein by reference in its entirety.

### GOVERNMENT SUPPORT

This invention was made with government support under Grant No. R01 CA226879 awarded by the National Institutes of Health and Grant No. T32 AI007405 awarded by the National Institutes of Health. The government has certain rights in the invention.

### FIELD OF INVENTION

The present invention relates generally to the fields of molecular biology, immunology, oncology and medicine. More particularly, it concerns immune cells that express granzyme protease(s), methods of producing and methods of using the immune cells for the treatment of cancer.

### BACKGROUND

Granzymes are a class of cytotoxic proteases produced in multiple immune cell subtypes, including CD8 T cells. Granzymes are produced in an inactive state and ultimately stored in the granules of cytotoxic immune cells where they are sequestered away from the cytosol before being activated. This protects the cells producing the granzyme from their cytotoxic activity. Upon reacting with a target cell, for example, a malignant cancer cell or infected cell, T cells release their granules towards target cells and other proteins stored in the granules, such as perforin, mediate entry of the granzyme proteases through the membrane of target cells allowing them to execute their cytotoxic function and eliminate target cells. Each granzyme acts on a unique set of substrates, most of which induce some form of cell death.

There are multiple forms of regulated cell death, the most common of which is apoptotic cell death which results in activation of the caspase cascade (Messmer et al. Cell Death and Differentiation 2019; 26 (1): 115-29). Apoptosis culminates in cellular blebbing, where cellular contents are released in an immunologically ‘quiet’ manner (Ferguson et al., Journal of immunology (Baltimore, Md: 1950) 2002; 168 (11): 5589-95; Fadok et al., The Journal of clinical investigation 1998; 101 (4): 890-8; Huang et al., Nature medicine 2011; 17 (7): 860-6). However, alternative forms of cell death, such as necroptosis, can induce potent immune responses through destruction of the cellular membrane and release of intracellular contents from dying cells (Green et al., Nature Reviews Immunology 2009; 9 (5): 353-63; Kaczmarek et al., Immunity 2013; 38 (2): 209-23). Some forms of cell death, such as pyroptosis, result in induction of inflammatory processes through the initiation of the inflammasome pathway (Aglietti et al., Proc Natl Acad Sci USA 2016; 113 (28): 7858-63; Ding et al., Nature 2016; 535 (7610): 111-6; Liu et al., Nature 2016; 535 (7610): 153-8).

Granzyme B induces canonical apoptotic cell death through direct cleavage of caspase-3 or through activation of the protein BID (Andrade et al., Immunity 1998; 8 (4): 451-60; Yang et al., The Journal of biological chemistry 1998; 273 (51): 34278-83; Sutton et al. J Exp Med 2000; 192 (10): 1403-14; Julien et al., Cell death and differentiation 2017; 24 (8): 1380-9; Tadokoro et al., Cell Death & Disease 2010; 1 (10): e89-e). Gzmb is one of the most highly expressed granzyme genes. Gzma, another highly expressed granzyme gene, induces pyroptosis through activation of gasdermin proteins (Zhou et al., Science (New York, NY) 2020; 368 (6494): eaaz7548), which may modulate the TME by increasing the inflammatory milieu. Gzmf expression is low in T cells under most conditions but induces a necroptotic-like form of cell death resulting in rupture of the cellular membrane (Shi et al. Cell Death and Differentiation 2009; 16 (12): 1694-706). Since necroptosis is a more immunogenic form of cell death, Gzmf expression may be a marker of a more tumoricidal T cell capable of eliciting epitope spread.

The consequence of granzyme expression on the T cell producing it is not clear. Given the cytotoxic nature of granzymes, T cells themselves may be susceptible to the cytotoxic activity of these proteases. Though there are mechanisms described which protect T cells from granzymes (Kaiserman et al. Cell Death & Differentiation 2010; 17 (4): 586-95), it has also been found that granzyme B induces apoptotic cell death in T cells that produce it (Devadas et al., Immunity 2006; 25 (2): 237-47). Since granzymes are potent mediators of cytotoxicity, better understanding of which granzymes are produced by T cells under different conditions may prove critical to improvement of cancer immunotherapies.

Chimeric antigen receptor (CAR) T cell therapy takes advantage of the cytotoxic pathways of granzyme-producing T cells to target cancers. When the recombinant antigen-binding receptor made from the variable region of a single-chain antibody binds to its antigen, a signal is transduced through the downstream activation domains and granzymes are produced and secreted by the T cells. CAR T cells targeting CD19 have shown remarkable clinical activity in patients with highly refractory B-lineage leukemias and lymphomas, however less than 50% of these patients will achieve a long-term remission. Furthermore, CAR T cell therapies for myeloid leukemias and solid tumors have not yet achieved similar levels of success. The heterogeneity of antigen expression is likely a barrier to CAR T cell success in such malignancies, and strategies which can induce epitope spreading may improve the effectiveness of CAR T cells for such patients.

Without wishing to be bound by theory, it is believed that by modulating the amount and ratio of granzymes produced in T cells, the cytotoxic capacity of adoptive transfer cell therapies, and even the form of cell death induced in target cells, can be altered (some forms of cell death are immune-stimulatory while others are immune-suppressive). In turn, modulating the cytotoxicity of transferred T cells as well as the type of cell death induced may improve the efficacy of these cell therapies by improving the capacity of the transferred cells themselves, but also by modulating the immunogenicity of the dying target cells. Modulating the tumor microenvironment through the form of cell death occurring in cancer cells is of particular interest given the suppressive tumor microenvironment during an anti-cancer immune response. This novel strategy for improving adoptive cell therapies, such as chimeric antigen receptor (CAR) T cell therapy, focuses directly on the terminal stage of immune-cell interaction with target cell, the induction of cell death. Elimination of target cells is the ultimate goal of these immune-therapies, and this is a new approach to achieve this goal, that could directly improve these therapies.

This disclosure describes is an improvement over modern T cell adoptive transfer, or CAR T cell therapies, which rely on the inherent cytotoxic capacity of the patients T cells. Engineered T cells with increased effective cytotoxic activity in the transferred cells will be produced by increasing production of specific granzymes or multiple specific granzymes. The current standard in adoptive transfer T cell therapies, which are clinically used, does not alter the cytotoxicity of the transferred cells. In one aspect, it is believed that altering in particular the terminal stage of T cell function, induction of target cell death, may improve the efficacy of these treatments in a way that has not yet been pursued.

### SUMMARY

In one aspect, provided herein is a modified CAR cell comprising a chimeric antigen receptor (CAR) and an exogenous polynucleotide encoding at least one granzyme. In another aspect, provided herein is a modified CAR cell produced by introducing into the CAR cell an exogenous polynucleotide encoding at least one granzyme. In some embodiments, the granzyme is granzyme F. In some embodiments, the exogenous polynucleotide comprises (i) a MSCV promoter, (ii) a chimeric antigen receptor (CAR), (iii) at least one linker domain (L), and (iv) a granzyme gene (Gzm).

In some embodiments, the modified CAR cell is an immune cell. In some embodiments, the immune cell is a T-cell, a hematopoietic progenitor cell, a peripheral blood (PB) derived T-cell or an umbilical cord blood (UCB) derived T-cell. In some embodiments, immune cell is a CD8+ T cell. In some embodiments, the granzyme gene encodes granzyme A, granzyme B, or granzyme F.

In another aspect, provided herein is a method of improving a cell therapy, comprising contacting a target cell with a modified CAR cell provided herein, wherein the amount of granzyme produced by the modified CAR cell is increased compared to an unmodified CAR cell. In some embodiments, the increased granzyme production results in an increase in cell death in the target cell as compared to contacting the target cell with an unmodified CAR cell. In some embodiments, the increased granzyme production results in an altered ratio of granzymes produced by the modified CAR cell compared to an unmodified CAR cell. In some embodiments, the increased granzyme production results in increased cytotoxic capacity of the modified CAR cell compared to an unmodified CAR cell. In some embodiments, the modified CAR cell induces a different form of cell death in the target cell compared to an unmodified CAR cell. In some embodiments, the form of cell death of the target cell is caspase-independent and/or results in rupture of the target cell membrane. In some embodiments, the form of cell death of the target cell is non-apoptotic. In some embodiments, the target cell is a cancer cells. In some embodiments, the cell therapy is a method of treating cancer.

### BRIEF DESCRIPTION OF THE DRAWINGS

FIG. 1 shows a schematic depicting an exemplary embodiment of a granzyme expressing T cell and method of use described herein.

FIGS. 2A-2E show that expression of Gzmf in CD8 T cells is higher in the TME than other tissues in multiple tumor models. Microarray data from Gene Expression Omnibus (GEO) was examined for expression of granzyme encoding genes in CD8 TIL relative to CD8 T cells from either matched spleens or lymph nodes. T cells were analyzed from the CT26 tumor (FIG. 2A; n=3), the MC38 tumor (FIG. 2B; n=2) and the B16 tumor (FIG. 2C; n=3). Microarray data were analyzed using the Geo2r software, and only significantly altered genes were shown as determined by a corrected P-value<0.05. Further explanation of the data is in the original publications. FIGS. 2D and 2E show quantitative PCR (qPCR) performed for multiple granzymes genes, Prf1 and Fasl expression in CD8 TIL and CD8 T cells from the spleen. Relative expression of these genes (left), and transcripts per 100,000 housekeeping transcripts in the spleen (middle) and tumor (right), are shown in the CT26 tumor model (FIG. 2D; n=5) and the A223 tumor model (FIG. 2E; n=5). For qPCR data a one sample t test was used to compare the fold change for each gene to no change in expression. For all genes with a P-value<0.05, a single star indicates significance.

FIGS. 3A-3G show how expression of the granzyme genes is differentially regulated after TCR stimulation. T cells from the spleens of naïve mice were stimulated with anti-CD3 and anti-CD28 beads (CD3/CD28) and gene expression was measured 3 days and 5 days later by qPCR for Gzma (FIG. 3A), Gzmb (FIG. 3B), Gzmf (FIG. 3C), Gzmk (FIG. 3D) and Gzmm (FIG. 3E) relative to unstimulated controls. Statistical significance was determined by a paired t test. FIG. 3F shows the fold change in granzymes, Prf1 and Fasl gene expression 5 days post stimulation, relative to matched unstimulated CD8 splenocytes, is shown. A one-sample t test was used to compare fold change values to no change in expression. FIG. 3G shows the expression of Gzma, Gzmb, Gzmf, Gzmk, Gzmm, Prf1 and Fasl was determined under different in vitro conditions, stimulated with either CD3/CD28 alone, Interferon-gamma (IFNγ), TGFbeta, or tumor-conditioned medium (TCM). All samples received CD3/CD28 for the first 3 days and 40 U/mL IL-2 while in culture. Statistical significance between the fold change in gene expression between the different treatment groups was determined by ANOVA. (n=6 for all samples except for TGF-beta treated cells where n=4, p≤0.05*, ≤0.01**, ≤0.001***, ≤0.0001****).

FIGS. 4A-4E show that Gzmf is expressed more frequently in exhausted PD1+TIM3+CD8 TIL than CD8 splenocytes or PD1-TIL. Using PrimeFlow analysis, the frequency of total CD8 T cells that express Gzmf from the TME or from the spleen was determined (FIG. 4A; n=4). Gzmf+ cells were examined for expression of the antigen-experienced marker 4-1BB (FIG. 4B; n=6) and co-expression of exhaustion markers PD1 and TIM3 (FIG. 4C; n=7). PrimeFlow analysis shows a subset of PD1 and TIM3 double-positive cells expressed Gzmf (FIG. 4D; n=7). FIG. 4E shows representative flow plots show PD1 and TIM3 expression of Gzmf+ cells (blue) and of bulk CD8 TIL (red). A t test was used to determine statistical significance between splenocyte and TIL expression of Gzmf, and between Gzmf+ and Gzmf-TIL expression of 4-1BB. A repeated measures ANOVA was used to determine statistical significance in the expression of PD1 and TIM3 in Gzmf+ cells (p≤0.05*, ≤0.01**, ≤0.001***, ≤0.0001****).

FIGS. 5A-5C depict an exemplary method using tetramer binding and single cell RNA sequencing to determine differentiation of antigen-specific CD8 TIL in mouse tissue. T cells are isolated from tumor bearing mice and sorted based on tetramer binding. FIG. 5B depicts clustering of antigen-specific CD8 TIL by uniform manifold approximation and projection (UMAP) analysis. FIG. 5C shows a series of UMAP plots depicting RNA profiles of CD8 TIL.

FIGS. 6A-6D show a series of UMAP analysis plots depicting clustering of Gmz gene expression using the differentiation method depicted in FIG. 5A. FIG. 6A shows clustering of T cells expressing GzmA. FIG. 6B shows clustering of T cells expressing GzmB. FIG. 6C shows clustering of T cells expressing GzmF. FIG. 6D shows clustering of T cells expressing Tim3.

FIG. 7 shows a schematic depicting an exemplary method of constructing T cells expressing a chimeric antigen receptor (CAR) and Gzm genes. Plasmids containing CARs and individual Gzm are introduced into T cells.

FIG. 8 shows a bar graph depicting Gzm expression in CAR T cells after stimulation relative to unstimulated CD3 cells. The x-axis depicts the gene measured. The y-axis depicts fold-change in expression.

FIG. 9 shows a bar graph depicting Gzm expression in CAR T cells relative to untransduced CD3 cells. The x-axis depicts the gene measured. The y-axis depicts fold-change in expression.

FIGS. 10A-B show a series of graphs depicting cytotoxicity capacity of Gzm over-expressing CARs relative to plain CARs and untransduced T cells. FIG. 10A shows E2a-GFP (target) cell count/(killing). The x-axis indicates the cell population. The y-axis indicates the E2a-GFP+ cell count. FIG. 10B shows the ratio of E2a cells to CAR cells. The x-axis depicts the cell type. The y-axis depicts the E2a/CAR.

FIG. 11 shows a bar graph depicting the GFP+E2a (target) cell death status. The x-axis depicts target cell status. The y-axis depicts the percent (frequency) of remaining E2a cells.

FIGS. 12A-12D show that F CARs have improved tumoricidal activity and induce an alternative form of cell death in target cancer cells relative to other granzyme overexpressing CARs. E2a-GFP cells were cocultured with CAR T cells at a 1:2 effector to target (E:T) ratio overnight. The ratio shown of E2a-GFP cells to CAR T cells after the co-culture was determined by flow cytometry (FIG. 12A). The frequency of viable E2a-GFP cells is shown as a percentage of the total E2a-GFP population (FIG. 12B). The percentage of dying E2a-GFP cells (sum of Annexin V+ and permeable membrane dye+ cells) is shown, which were positive for Annexin V and negative for the permeable membrane dye (FIG. 12C), or positive for the permeable membrane dye and negative for Annexin V (FIG. 12D). A repeated measures ANOVA was used to determine statistical significance between all experimental groups (n=10 for all experimental groups and was repeated 5 times, p≤0.05*, ≤0.01**, ≤ 0.001***, ≤0.0001****).

FIGS. 13A-13D show that F CARs have reduced viability relative to other CAR groups and granzyme overexpressing CARs have reduced CD69 expression in vitro. FIG. 13A: After an overnight co-culture of CAR T cells with E2a-GFP cells (E:T=1:2), the frequency of CAR T cell death was normalized to CARnegative T cell death in the same culture. FIG. 13B: The frequency of dying cells that had a permeable membrane (orange bars) or were only annexin V positive (blue bars) is shown. The gMFI of CD69 expression as determined by flow cytometry after E2a-GFP co-culture is shown for CAR+ T cells (FIG. 13C) and the difference between CAR expressing and untransduced T cells (FIG. 13D). A repeated measures ANOVA was used to determine statistical significance. (n=10 for all experimental groups and was repeated 5 times, p≤0.05*, ≤0.01**, ≤0.001***, ≤0.0001****).

FIGS. 14A-14E show that plain CARs and B CARs control antigen-expressing tumor cell growth better than F CARs and A CARs. The frequency of CARs in the bone marrow 55 days after CAR transfer as a percentage of total T cells (FIG. 14A), CD4 T cells (FIG. 14B), and CD8 T cells (FIG. 14C) independently. The frequency of CAR T cells in circulation 5 days post CAR transfer is shown as a percentage of T cells. ANOVA (FIG. 14D) was used to determine statistical significance. CD19+ cell-free survival, defined as less than 1% of viable lymphocytes expressing CD19 and B220, after E2a cancer cell challenge is shown in a Kaplan-Meyer survival plot (FIG. 14E) and evaluated for statistical significance by a log rank test; a Bonferonni corrected alpha value was used to correct for multiple comparison bias, and n=10 mice per group.

### DETAILED DESCRIPTION

The present invention generally provides a method of improving an adoptive cell therapy comprising chimeric antigen receptor (CAR) cells, including immune cells (e.g., T cells, B cells, Natural Killer (NK) cells, monocytes, macrophages or artificially generated cells with immune effector function) derived from a patient, a healthy donor, a differentiated stem cell (including but not limited to induced pluripotent stem cells (iPSC), embryonic stem cells, hematopoietic and/or other tissue specific stem cells) or a non-human source, which are genetically modified to overproduce at least one granzyme, and methods of use thereof for the treatment of cancer.

The present invention provides an immune cell (e.g. T cell) expressing at least one granzyme and a chimeric antigen receptor (CAR) comprising: (a) an ectodomain comprising an antigen recognition region; (b) a transmembrane domain; (c) at least one costimulatory domain; and (d) an intracellular signaling domain. In some embodiments, the immune cell is a T cell. In some embodiments, the T cell is a CD8⁺ T cell.

The present disclosure overcomes problems associated with current technologies (e.g., CAR T cell therapies) by providing therapeutic immune cells (e.g. T cells) such as for the treatment of cancer or infectious disease. The present disclosure represents the first discovery and the first use of immune cells (e.g. T cells) expressing chimeric antigen receptors and overproducing at least one granzyme for improved cytotoxicity and modulated killing of target cells. The present disclosure is based, at least in part, on the discovery that immune cell (e.g. T-cell) 1) overproduction of at least one granzyme results in increased cellular cytotoxicity and 2) overproduction of at least one specific lowly expressed granzymes alters the form of cell death induced by T cells. Accordingly, the present disclosure provides immune cells expressing CARs and at least one granzyme, and methods of generating the cells and methods of using this population of cells.

Genetic reprogramming of immune cells (e.g. T cells), for adoptive cancer immunotherapy has clinically relevant applications and benefits such as 1) increased cytotoxic capacity and elimination of target cells of cell therapy, 2) altered form of cell death induced in target cells, 3) altered immunogenicity of dying target cells and 4) modulation of the tumor microenvironment. Accordingly, the present disclosure also provides methods for treating cancer, comprising adoptive cell immunotherapy with any of the engineered immune cells provided herein.

A CAR generally comprises an extracellular ectodomain comprising an antigen-binding recognition region (e.g., an antibody or a part of an antibody such as a single chain variable fragment (scFv)), a transmembrane domain, and an intracellular domain. The intracellular domain often comprises a CD33 signaling domain and one or two costimulatory domains, which may be derived from, for example, CD8, CD28, OX40 or 4-1BB, or be a combination of the same. See Jayaraman et al., EBioMedicine 58 (2020) 102931, which is incorporated herein by reference in its entirety.

In some embodiments, the CD3ζ domain is linked or fused to the α chain of a TCR. In some embodiments, the CD3ζ domain is linked or fused to the β chain of a CDR. In some embodiments, one CD3ζ subunit is linked to each of the α chain and the β chain of the TCR.

### Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “about” is used to indicate that a value includes the inherent variation of error for the device, the method being employed to determine the value, or the variation that exists among the study subjects.

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. In some embodiments, if a full-length polypeptide binds a given ligand, a portion of that full-length polypeptide also binds to the same ligand.

The terms “protein” and “polypeptide” are used interchangeably herein.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide, or the protein expressed by said polynucleotide, to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, that when present in a plasmid in a cell is capable of maintaining linked sequences in the plasmid and/or a site at or near where DNA synthesis initiates. As an example, an ori for EBV (Ebstein-Barr virus) includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, methods of the present disclosure may also use genetically engineered replication origin of EBV, such as by insertion or mutation of individual elements.

A “gene,” “polynucleotide,” “coding region,” “sequence,” “segment,” “fragment,” or “transgene” that “encodes” a particular protein, is a section of a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein to refer to a nucleotide region comprising a DNA regulatory sequence, wherein the regulatory sequence is derived from a gene that is capable of binding to a RNA polymerase and allowing for the initiation of transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and an functional effector element) are connected in such a way as to permit transcription of the nucleic acid molecule. “Operably linked” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of molecules that are not found in a single polypeptide in nature.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the term “subject” or “subject in need thereof refers to a mammal, preferably a human being, male or female at any age that is in need of a therapeutic intervention, a cell transplantation or a tissue transplantation. Typically, the subject is in need of therapeutic intervention, cell or tissue transplantation (also referred to herein as recipient) due to a disorder or a pathological or undesired condition, state, or syndrome, or a physical, morphological or physiological abnormality which is amenable to treatment via therapeutic intervention, cell or tissue transplantation.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a NK cell, B cell, or a T cell, or innate immune cell to a stimulus. In one embodiment, the response is specific for a particular antigen (an “antigen-specific response”).

As used herein, the term “antigen” is a molecule capable of being bound by an antibody, T-cell receptor, Chimeric Antigen Receptor and or engineered immune receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

An “epitope” is the site on an antigen recognized by an antibody as determined by the specificity of the amino acid sequence. Two antibodies are said to bind to the same epitope if each competitively inhibits (blocks) binding of the other to the antigen as measured in a competitive binding assay. Alternatively, two antibodies bind to the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

A “parameter of an immune response” is any particular measurable aspect of an immune response, including, but not limited to, cytokine secretion (IFN-γ, etc.), chemokine secretion, altered migration or cell accumulation, immunoglobulin production, dendritic cell maturation, regulatory activity, number of immune cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of 3H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

“Treating” or “treatment of” a disease or condition refers to executing a protocol or treatment plan, which may include administering one or more drugs to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. Alleviation or prevention can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and includes protocols or treatment plans that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis or recurrence. Treatment of cancer may also refer to prolonging survival of a subject with cancer.

“Antigen recognition moiety” or “antigen recognition domain” refers to a molecule or portion of a molecule that specifically binds to an antigen. In one embodiment, the antigen recognition moiety is an antibody, antibody like molecule or fragment thereof and the antigen is a tumor antigen.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHL CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

“Antibody like molecules” may be for example proteins that are members of the Ig-superfamily which are able to selectively bind a partner.

The terms “fragment of an antibody,” “antibody fragment,”, “functional fragment of an antibody,” and “antigen-binding portion” are used interchangeably herein to mean one or more fragments or portions of an antibody that retain the ability to specifically bind to an antigen (see, generally, Holliger et al. (2005) Nat. Biotech. 23 (9): 1126-29). The antibody fragment desirably comprises, for example, one or more CDRs, the variable region (or portions thereof), the constant region (or portions thereof), or combinations thereof.

Examples of antibody fragments include, but are not limited to, (i) a Fab fragment, which is a monovalent fragment consisting of the VL, VH, CL, and CH1 domains; (ii) a F (ab′) 2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al. (1988), Science 242:423-6; Huston et al. (1988) Proc. Natl. Acad. Sci. USA 85:5879-83; and Osbourn et al. (1998) Nat. Biotechnol. 16:778-81) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1.

A “chimeric antigen receptor” is also known as an artificial cell receptor, a chimeric cell receptor, or a chimeric immunoreceptor. Chimeric antigen receptors (CARs) are engineered receptors, which graft a selected specificity onto an immune effector cell. CARs typically have an extracellular domain (ectodomain), a transmembrane domain and an intracellular (endodomain) domain. In some embodiments, the ectodomain comprises an antigen-binding domain and a stalk region. In some embodiments, the ectodomain comprises an antibody binding domain that recognizes CD19.

A “stalk region”, which encompasses the terms “spacer region” or “hinge domain” or “hinge”, is used to link the antigen-binding domain to the transmembrane domain. As used herein, the term “stalk region” generally means any oligonucleotide or polypeptide that functions to link the transmembrane domain to, either the extracellular domain or, the cytoplasmic domain in the polypeptide chain of a CAR. In embodiments, it is flexible enough to allow the antigen-binding domain to orient in different directions to facilitate antigen recognition.

The term “functional portion,” when used in reference to a CAR, refers to any part or fragment of a CAR described herein, which part or fragment retains the biological activity of the CAR of which it is a part (the parent CAR). In reference to a nucleic acid sequence encoding the parent CAR, a nucleic acid sequence encoding a functional portion of the CAR can encode a protein comprising, for example, about 10%, 25%, 30%, 50%, 68%, 80%, 90%, 95%, or more, of the parent CAR.

The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “T cell” refers to T lymphocytes, and includes, but is not limited to, γ/δ T cells, α/β T cells, NK T cells, CD4⁺ T cells and CD8⁺ T cells. CD4⁺ T cells include THO, Tₕ1 and TH2 cells, as well as regulatory T cells (T_{reg}). There are at least three types of regulatory T cells: CD4⁺ CD25⁺ T_{reg}, CD25 T_{H}3 T_{reg}, and CD25 T_{R}1 T_{reg}. “Cytotoxic T cell” refers to a T cell that can kill another cell. The majority of cytotoxic T cells are CD8⁺ MHC class I-restricted T cells, however some cytotoxic T cells are CD4⁺. In some embodiments, the T cell of the present disclosure is CD4⁺ or CD8⁺.

“Tumor antigen” as used herein refers to any antigenic substance produced, expressed or overexpressed in tumor cells. It may, for example, trigger an immune response in the host.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B cells, endothelial cells, activated T cells, and dendritic cells; or other molecules, naturally occurring or synthetic, such as purified MHC Class I molecules complexed to 2-microglobulin.

The term “culturing” refers to the in vitro maintenance, differentiation, and/or propagation of cells in suitable media. By “enriched” is meant a composition comprising cells present in a greater percentage of total cells than is found in the tissues where they are present in an organism.

### Immune Cells

Certain embodiments of the present disclosure concern immune cells which express a chimeric antigen receptor (CAR). The immune cells may be T cells (e.g., regulatory T cells, CD4+ T cells, CD8+ T cells, or gamma-delta T cells), NK cells, invariant NK cells, NKT cells, stem cells (e.g., mesenchymal stem cells (MSCs) or induced pluripotent stem (iPSC) cells). In some embodiments, the T cell is a tumor infiltrating lymphocyte (TIL). In some embodiments, the cells are monocytes or granulocytes, e.g., myeloid cells, macrophages, neutrophils, dendritic cells, mast cells, eosinophils, and/or basophils. Also provided herein are methods of producing and engineering the immune cells and methods of using and administering the cells for adoptive cell therapy, in which case the cells may be autologous or allogeneic. Thus, the immune cells may be used as immunotherapy, such as to target cancer cells.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. Thus, the cells will be autologous to the subject in need of therapy. Alternatively, the immune cells can be obtained from a donor and therefore be allogeneic to the subject in need of therapy.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. The isolated immune cells may be used directly, or they can be stored for a period of time, such as by freezing. In some embodiments, the immune cells are isolated from blood, such as peripheral blood or cord blood. In some embodiments, immune cells isolated from cord blood have enhanced immunomodulation capacity, such as measured by CD4-positive or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood. The population of immune cells can be derived from induced pluripotent stem cells (iPSCs) and/or any other stem cell known in the art. In some aspects, the iPSCs and/or stem cells used to derive the population of immune cells can be obtained from a subject in need of therapy or suffering from a disease associate with reduced immune cell activity, thus these IPSCs and/or stem cells will be autologous to the subject in need of therapy. Alternatively, the iPSCs and/or stem cells can be obtained from a donor and therefore be allogeneic to the subject in need of therapy.

When the population of immune cells is obtained from a donor distinct from the subject, the donor is preferably allogeneic, provided the cells obtained are subject-compatible in that they can be introduced into the subject. Allogeneic donor cells are may or may not be human leukocyte antigen (HLA)-compatible. To be rendered subject-compatible, allogeneic cells can be treated to reduce immunogenicity.

### T Cells

T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. There are six types of T-cells, namely: Helper T-cells (e.g. CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory TSCM cells, like naive cells, are CD45RO−, CCR7+, CD45RA+, CD62L+ (L-selectin), CD27+, CD28+ and IL-7Ra+, but they also express large amounts of CD95, IL-2R, CXCR3, and LFA-1, and show numerous functional attributes distinctive of memory cells); (ii) central memory TCM cells express L-selectin and the CCR7, they secrete IL-2, but not IFNg or IL-4, and (iii) effector memory TEM cells, however, do not express L-selectin or CCR7 but produce effector cytokines like IFNg and IL-4), Regulatory T-cells (Tregs, suppressor T cells, or CD4+CD25+ regulatory T cells), Natural Killer T-cells (NKT) and Gamma Delta T-cells.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

### Genetically Engineered Chimeric Antigen Receptors

The immune cells of the disclosure (e.g., autologous or allogeneic T cells (e.g., regulatory T cells, CD4⁺ T cells, CD8⁺ T cells, or gamma-delta T cells), NK cells, invariant NK cells, NKT cells, stem cells (e.g., MSCs or iPS cells) can be genetically engineered to express antigen receptors such as engineered CARs. In particular embodiments, T cells are engineered to express a CAR. Multiple CARs, may be added to a single cell type, such as T cells.

In some embodiments, the cells comprise one or more nucleic acids introduced via genetic engineering that encode one or more antigen receptors, and genetically engineered products of such nucleic acids. In some embodiments, the nucleic acids are heterologous. In some embodiments, the nucleic acids are not naturally occurring, such as a nucleic acid not found in nature (e.g., chimeric).

In some embodiments, the CAR contains an extracellular antigen-recognition domain that specifically binds to an antigen (e.g., a cancer cell, or an infected cell). In some embodiments, the antigen is a protein expressed on the surface of cells (e.g., on the surface of a cancer cell, or an infected cell).

Exemplary engineered antigen receptors, including CARs, as well as methods for engineering and introducing the receptors into cells, include those described, for example, in PCT Publication Nos. WO 2000/14257, WO 2013126726, WO 2012/129514, WO 2014/031687, WO 2013/166321, WO 2013/071154, and WO 2013/123061, U.S. Patent Application Publication Nos. US 2002/131960, US 2013/287748, and US 2013/0149337; and U.S. Pat. Nos. 6,451,995, 7,446,190, 8,252,592, 8,339,645, 8,398,282, 7,446,179, 6,410,319, 7,070,995, 7,265,209, 7,354,762, 7,446,190, 7,446,191, 8,324,353, and 8,479, 118; International Patent Application Publication No.: WO 2014/055668 A1, and European Patent Application Publication No. EP2537416; and/or those described by Sadelain et al., 2013; Davila et al., 2013; Turtle et al., 2012; Wu et al., 2012.

In some aspects, the present disclosure provides a population of genetically modified immune cells (e.g. T cells) engineered to express a chimeric antigen receptor (CAR) and/or a polynucleotide encoding a CAR, wherein the CAR comprises (a) an ectodomain comprising an antigen recognition region; (b) a transmembrane domain; (c) at least one costimulatory domain; and (d) an intracellular signaling domain.

In some embodiments, the genetically engineered cells include additional CARS, including activating or stimulatory CARs, co-stimulatory CARs (see, e.g., PCT Publ. No. WO 2014/055668), and/or inhibitory CARs (iCARs, see, e.g., Fedorov et al., 2013). The CARs generally include an extracellular antigen (or ligand) recognition domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone. For example, once an antigen is recognized by the ectodomain, the intracellular signaling components transmit an activation signal to the T cell that induces the T cell to destroy a targeted tumor cell.

The present disclosure provides a population of engineered T cells, wherein a plurality of the engineered T cells of the population comprise any chimeric stimulatory receptor (CAR) disclosed herein. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the CAR. In some embodiments, each CAR polypeptide is expressed at a copy number of at least 1, 2, 3, 4, 5, 6, 7, 8, 9, 10, 20, 30, 40, 50, 60, 70, 80, 90 or 100 copies per cell. In some embodiments, the nucleic acid encoding the CAR is integrated into the genome at a copy number of at least 1, 2, 3, 4, 5, 6, 7, 8, 9, 10, 20 or 30 copies per cell. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the CAR and an exogenous polynucleotide encoding at least one granzyme.

### Target Antigens

Provided herein are immune cells (e.g., T cells) expressing a CAR that targets a cancer cell. In some embodiments, the cancer cell is located in a tumor. In some embodiments, the cancer cell is not located in a tumor.

In some embodiments, the immune cells (e.g. T cells) expressing a CAR target an infected cell. In some embodiments, the infected cell is an infected host cell. In some embodiments, the infected cell is an infected host immune cell.

Among the antigens that may be targeted by the genetically engineered antigen receptors are those expressed in the context of a disease, condition, or cell type to be targeted via the adoptive cell therapy. Among the diseases and conditions are aberrant or misregulated immune responses such as cancers, autoimmune disorders, diseases of immunity, and conditions characterized by chronic inflammation. Aberrant or pathological immune activation underlies diseases, such as autoimmune diseases, solid transplant rejection, transplantation graft rejection, allergy, asthma, diabetes mellitus and rheumatoid arthritis and T cell leukemia.

In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary target antigens include, but are not limited to, antigens expressed on the surface of cancer cells or infected cells described herein.

### T Cell Activity

In some embodiments, a population of genetically engineered T cells as disclosed herein exhibits T cell functions (e.g., effector functions). In some embodiments, the population is cytotoxic to cancer cells. In some embodiments, the population is cytotoxic to infected cells. Effector function of a genetically engineered T cell, for example, may be cytolytic activity or helper activity including the secretion of cytokines. In some embodiments, the population exhibits one or more T cell effector functions at a level that is least 2-3-fold, at least 3-4-fold, at least 4-5-fold, at least 5-10-fold, at least 10-15-fold, at least 15-20-fold, or more than 20-fold higher than the functions exhibited by a population of T cells not expressing the CAR.

### Granzymes

Granzymes (Gzms), a family of cytotoxic proteases, are key effector molecules used by cytotoxic T lymphocytes (CTLs) to eliminate target cells. Gzms are the primary mechanism utilized by T cells to directly eliminate target tumor cells. The direct elimination of target cells is a key function of Gzms, and each Gzm acts upon unique substrates in target cells to induce cytoxicity through a range of mechanisms. Gzms can be expressed by multiple different immune cell types and the expression pattern of each Gzm within different cell types differs. In some embodiments, a therapeutic cell (e.g., a T cell) as disclosed herein overproduces at least one granzyme. The granzyme may be expressed by the cell prior to the introduction into said cell of an exogenous polypeptide expressing said at least one granzyme. In some embodiments, the at least one granzyme isgranzyme A, granzyme B, granzyme F, or any combination thereof.

In some embodiments, one granzyme is produced from two genetic loci in the therapeutic cell. In some embodiments, one granzyme is produced from at least two genetic loci in the therapeutic cell. In some embodiments, the at least one granzyme is expressed from an exogenous polynucleotide. In some embodiments, the therapeutic cell expresses a granzyme from the genomic DNA and from an exogenous polynucleotide. In some embodiments, a therapeutic cell overproduces at least one granzyme not expressed in the cell prior to introduction into said cell of an exogenous polynucleotide expressing said at least one granzyme. In some embodiments, a granzyme is produced from one genetic locus in the therapeutic cell. In some embodiments, one granzyme is produced from at least one genetic locus in the therapeutic cell.

In some embodiments, a therapeutic cell as disclosed herein overproduces one granzyme. In some embodiments, a therapeutic cell as disclosed herein overproduces at least one granzyme. In some embodiments, a therapeutic cell as disclosed herein overproduces at least two granzymes. In some embodiments, a therapeutic cell as disclosed herein overproduces at least three granzymes.

In some embodiments, the overproduction of at least one granzyme increases the cytotoxic capacity of the therapeutic cell. In some embodiments, a single therapeutic cell kills a larger amount of target cancer cells. In some embodiments, a population of therapeutic cells kill a larger amount of target cancer cells. In some embodiments, the overproduction of at least one granzyme alters the ratio of individual granzymes in the therapeutic cell. In some embodiments, the overproduction of at least one granzyme alters the form of cell death induced by the therapeutic cell.

### Methods of Improving Cell Therapies

In another aspect, provided herein are methods of improving a cell therapy, e.g., an adoptive cell therapy. Such methods may comprise a step of introducing into a therapeutic cell an exogenous polynucleotide which encodes at least one granzyme. In some embodiments, a method of improving a cell therapy may result in a therapeutic cell overexpressing the at least one granzyme, e.g., overexpressing the at least one granzyme in comparison to the cell prior to the introduction of the exogenous polynucleotide. In some embodiments, the at least one granzyme is Granzyme A, Granzyme B, Granzyme F, or a combination thereof.

In some embodiments, a therapeutic cell comprising an exogenous polynucleotide encoding at least one granzyme further comprises a chimeric antigen receptor (CAR). CARs may be readily inserted into and expressed by immune cells, (e.g., T cells). In certain embodiments, cells (e.g., immune cells such as T cells) are obtained from a donor subject. In some embodiments, the donor subject is human patient afflicted with cancer. In other embodiments, the donor subject is a human patient not afflicted with cancer. In some embodiments, an engineered cell is autologous to a subject. In some embodiments, an engineered cell is allogeneic to a subject. Preferably, the methods of improving therapeutic cells and therapeutic populations of cells do not substantially affect the antigen specificity of the receptor comprised by the cells. Thus, in preferred embodiments, a method of improving a therapeutic cell described herein results in an improved cell with substantially the same antigen specificity. “Substantially the same antigen specificity” means that the type of antigen recognized by the receptor is unchanged and that the affinity of a receptor for the antigen is decreased by at most 10% compared to the receptor of the cell prior to improvement.

In some embodiments, the therapeutic cell produces an increased amount of the at least one granzymes relative to the therapeutic cell prior to improvement. For example, in some embodiments, a method of improving a cell therapy described herein results in a 1-2-fold, 2-3-fold, 3-4-fold, 4-5-fold, 5-10-fold, 10-15-fold, 15-20-fold, or more than 20-fold increase in the expression of at least one granzyme compared to the expression of the granzyme prior to the improvement. Granzyme expression levels may be determined using any suitable method known in the art or described herein.

In some embodiments, a method of improving a cell therapy described herein results in the production of an altered ratio of granzymes (e.g., the ratio of granzyme F to granzyme A, or the ratio of granzyme F to granzyme B) relative to the therapeutic cell prior to improvement.

In some embodiments, a method of improving a cell therapy described herein results in an increase in the cytotoxic capacity of the therapeutic cell relative to the therapeutic cell prior to improvement. In some embodiments, a method of improving a cellular therapy described herein results in a 1-2-fold, 2-3-fold, 3-4-fold, 4-5-fold, 5-10-fold, 10-15-fold, 15-20-fold, or more than 20-fold increase in cytotoxicity relative to the therapeutic cell prior to improvement. The cytotoxicity of a therapeutic cell may be measured using any suitable method known in the art or described herein.

In some embodiments, a method of improving a cell therapy described herein results in a change in the mechanism by which a therapeutic cell induced cell death in its target cell. For example, the method result in more non-apoptotic cell death compared to the therapeutic cell prior to improvement. In some embodiments, the methods results in more caspase-independent cell death compared to the therapeutic cell prior to improvement. The mechanism of cell death may be determined using any suitable method known in the art or described herein.

The cell of the present disclosure may be obtained through any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. Additional methods of isolating T cells for a T cell therapy are disclosed in U.S. Patent Publication No. 2013/0287748, which is herein incorporated by references in its entirety.

In some embodiments, PBMCs are used directly for genetic modification with the immune cells (such as CARs) using methods as described herein. In certain embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8+ cells are further sorted into naive, central memory, effector memory and effector cells by identifying cell surface antigens that are associated with each of these types of CD8+ cells. In certain embodiments, CD4+ T cells are further sorted into subpopulations. For example, CD4+ T helper cells can be sorted into naive, central memory, and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the exogenous polynucleotide encoding the at least one granzyme and/or the chimeric antigen receptors described herein and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

### Gene Delivery and Cell Modification

Expression cassettes included in vectors useful in the present disclosure contain (in a 5′-to-3′ direction) a transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

### Promoter/Enhancers

The expression constructs provided herein comprise a promoter to drive expression of the CAR and/or the at least one granzyme. A promoter used in the context of the present disclosure includes constitutive, inducible, and tissue-specific promoters. A promoter generally comprises a sequence that functions to position the start site for RNA synthesis. Additional promoter elements regulate the frequency of transcriptional initiation. The spacing between promoter elements frequently is flexible, so that promoter function is preserved when elements are inverted or moved relative to one another.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (ere), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is EF1, EF1alpha, MND, CMV IE, dectin-1, dectin-2, human CD1 1c, F4/80, SM22, RSV, SV40, Ad MLP, beta-actin, MHC class I, MHC class II promoter, U6 promoter or H1 promoter, however any other promoter that is useful to drive expression of the therapeutic gene is applicable to the practice of the present disclosure.

### Initiation Signals and Linked Expression

A specific initiation signal also may be used in the expression constructs provided in the present disclosure for efficient translation of coding sequences. These signals include the ATG initiation codon or adjacent sequences. One of ordinary skill in the art would readily be capable of determining this and providing the necessary signals.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages.

Additionally, certain 2A sequence elements could be used to create linked- or co-expression of genes in the constructs provided in the present disclosure. For example, cleavage sequences could be used to co-express genes by linking open reading frames to form a single cistron. Exemplary cleavage sequences include but are not limited to T2A, P2A, E2A and F2A. In a preferred embodiment, the cleavage sequence comprises a P2A sequence.

### Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

### Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers.

In addition to markers conferring a phenotype that allows for the discrimination of transformants based on the implementation of conditions, other types of markers including screenable markers such as GFP, whose basis is colorimetric analysis, are also contemplated. In some embodiments, the reporter genes such as tEGFR are used. Further examples of selection and screenable markers are well known to one of skill in the art. One of skill in the art would also know how to employ immunologic markers, possibly in conjunction with FACS analysis. The marker used is not believed to be important, so long as it is capable of being expressed simultaneously with the nucleic acid encoding a gene product.

Cells may be modified to express a modified receptors and/or at least one granzyme by any suitable method known in the art or described herein, for example, electroporation or lipofection. In some embodiments of the methods of the disclosure, introducing a nucleic acid sequence and/or a genomic editing construct into an immune cell ex vivo, in vivo, in vitro or in situ comprises a viral vector. In some embodiments of the methods of the disclosure, introducing a nucleic acid sequence and/or a genomic editing construct into an immune cell ex vivo, in vivo, in vitro or in situ comprises a combination of vectors. Exemplary, non-limiting vector combinations include: viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enable genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

In addition to viral delivery of the nucleic acids encoding the antigen receptor, the following are additional methods of recombinant gene delivery to a given cell, (e.g. an NK cell) and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

In some embodiments of the methods of the disclosure, introducing a nucleic acid sequence and/or a genomic editing construct into an immune cell ex vivo, in vivo, in vitro or in situ comprises a non-viral vector. In some embodiments, the non-viral vector comprises a nucleic acid. In some embodiments, the non-viral vector comprises plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In some embodiments, the non-viral vector comprises a transposon of the disclosure.

In some embodiments of the methods of the disclosure, enzymes may be used to create strand breaks in the host genome to facilitate delivery or integration of the transgene. In some embodiments, enzymes create single-strand breaks. In some embodiments, enzymes create double-strand breaks. In some embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN) or zinc finger nucleases (ZFN). In some embodiments, break-inducing enzymes can be delivered to the cell encoded in DNA, encoded in mRNA, as a protein, as a nucleoprotein complex with a guide RNA (gRNA).

### Pharmaceutical Compositions

Also provided herein are pharmaceutical compositions and formulations comprising immune cells (e.g., T cells) and a pharmaceutically acceptable carrier. The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In some embodiments, a pharmaceutical composition comprises a dose ranging from about 1×10⁵ T cells to about 1×10⁹ T cells. In some embodiments, the dose is about 1×10⁵, 1×10⁶, 1×10⁷, 1×10⁸ or 1×10⁹ T cells. In some embodiments, a pharmaceutical composition comprises a dose ranging from about 5×10⁵ T cells to about 10×10¹² T cells.

### Methods of Treatment

In some embodiments, the present disclosure provides methods for immunotherapy comprising administering an effective amount of the immune cells (e.g. engineered T-cells) of the present disclosure. In some embodiments, a medical disease or disorder is treated by transfer of an immune cell population that elicits an immune response. In some embodiments, cancer is treated by transfer of an immune cell population that elicits an immune response. Provided herein are methods for treating or delaying progression of cancer in an individual comprising administering to the individual an effective amount an antigen-specific cell therapy. Diseases for which the present treatment methods are useful include any diseases wherein a pathologic, infected or cancer cell type is present in the subject.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of a cancer or which is capable of relieving symptoms caused by cancer, such as pain and inflammation. It can be the amount necessary to relieve symptoms associated with inflammation, such as pain, edema and elevated temperature.

The immune cell population can be administered in treatment regimens consistent with the disease, for example a single or a few doses over one to several weeks to ameliorate a disease state or periodic doses over an extended time to inhibit disease progression and prevent disease recurrence. The precise dose to be employed in the formulation will also depend on the route of administration, and the seriousness of the disease or disorder. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as from about 2×10⁷ cells to about 5×10⁸ cells per kg body weight, or from about 5×10⁷ cells to about 2×10⁸ cells per kg body weight, or from about 5×10⁶ cells per kg body weight to about 1×10⁷ cells per kg body weight. In some embodiments, a therapeutically effective amount of immune cells ranges from about 1×10⁵ cells per kg body weight to about 10×10⁹ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

In some embodiments, the method of improving a therapeutic cell results in a beneficial change in the tumor microenvironment. Examples of beneficial changes in the tumor environment are known in the art and include and is not limited to, for example, decreases in hypoxia and decreases in vascularization. See, e.g., Benavente et al., Front. Oncol., 23 Oct. 2020, which is incorporated herein by reference in its entirety.

The cells and pharmaceutical compositions described herein may be used to treat a disorder or disease in a subject in need thereof. Thus, in another aspect, provided herein is a method of treating cancer in a subject, comprising administering to the subject a cell comprising a chimeric antigen receptor overproducing at least one granzyme provided herein. In some embodiments, the disease or disorder is cancer.

Tumors for which the present treatment methods are useful include any malignant cell type, such as those found in a solid tumor or a hematological tumor. In some embodiments, the cancer is a CD22-positive cancer. In some embodiments, the cancer has a low expression of CD22 (e.g. a CD22 low cancer). In some embodiments, the cancer is a CD19-positive cancer. In some embodiments, the cancer has a low expression of CD19 (e.g. a CD19 low cancer).

Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include but are not limited to tumors of the bone marrow, T or B cell malignancies, myeloid malignancies, leukemias, lymphomas, blastomas, myelomas. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; T lymphoblastic leukemia; T lymphoblastic lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); chronic myeloid leukemia, acute lymphoblastic leukemia (ALL); acute lymphoblastic lymphoma; acute myeloid leukemia (AML); myelodysplastic syndrome (MDS); myeloproliferative neoplasms; chronic myeloblasts leukemia; diffuse large B-cell lymphoma (DLBCL); peripheral T-cell lymphoma (PTCL); or anaplastic large cell lymphoma (ALCL).

Particular embodiments concern methods of treatment of leukemia. Leukemia is a cancer of the blood or bone marrow and is characterized by an abnormal proliferation (production by multiplication) of blood cells, usually immature white blood cells (leukocytes). It is part of the broad group of diseases called hematological neoplasms. Leukemia is a broad term covering a spectrum of diseases. Leukemia is clinically and pathologically split into its acute and chronic forms and/or by and the cell type of origin (myeloid or lymphoid). In some embodiments, the leukemia is an antigen-low leukemia. In some embodiments, the leukemia is a CD22-low leukemia.

In some embodiments, the cancer is breast cancer, sarcoma, melanoma, or lung cancer. The terms “subject” and “patient” are used interchangeably herein. In some embodiments, the subject treated in accordance with the methods described herein is a human patient, e.g., a human adult.

In some embodiments, the cells are obtained from a patient, modified ex vivo, expanded, and reinfused to the patient. The cells may be allogeneic or autologous to the patient.

A person of skill in the art will be able to determine the appropriate duration and dose of treatment for a cell comprising a CAR and an exogenous polynucleotide encoding at least one granzyme provided herein and/or for a pharmaceutical composition provided herein.

The cell comprising a CAR and an exogenous polynucleotide encoding at least one granzyme provided herein or the pharmaceutical composition provided herein may be administered by any suitable route of administration, including, for example, intravenous, intrathecal, intraocular, subcutaneous, intraperitoneal, intramuscular, intracerebral, intraventricular, or intratracheal administration. In preferred embodiments, the cell comprising a CAR and an exogenous polynucleotide encoding at least one granzyme provided herein or the pharmaceutical composition provided herein is administered intravenously.

In certain embodiments of the present disclosure, immune cells are delivered to an individual in need thereof, such as an individual that has cancer or an infection. The cells then enhance the individual's immune system to attack or directly attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, 8, 9, 10, 11, or 12 or more weeks.

The immune cells may be administered in combination with one or more other therapeutic agents for the treatment of the immune-mediated disorder. The immune cells may be administered before, during, after, or in various combinations relative to an additional cancer therapy, such as radiation therapy, chemotherapy, or immune therapy (e.g., immune checkpoint therapy). The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

The cell comprising a CAR and an exogenous polynucleotide encoding at least one granzyme provided herein or the pharmaceutical composition provided herein may be administered for any suitable duration, for example, until symptoms improve, or for a predetermined duration such as 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks.

The efficacy of a treatment for cancer may be assessed by any suitable method known in the art or described herein, including, for example, by monitoring tumor growth or measuring the time to tumor recurrence. In some embodiments, a method of treatment described herein induces tumor regression. In some embodiments, the tumor regresses by at least 10%, by at least 20%, by at least 30% within about one week, about 2 weeks, about one month, about 2 months, about 3 months, about 6 months, about 9 months, or about 12 months after the first administration of the bispecific binding agent or functional fragment thereof or of the pharmaceutical composition.

In some embodiments, a method of treatment described herein results in regression of the tumor to undetectability and delays tumor recurrence. In some embodiments, the tumor recurrence is delayed by at least about 3 months, at least about 6 months, at least about 9 months, at least about 12 months, at least 18 months, at least 24 months, at least 3 years, at least 4 years or at least 5 years after the tumor becomes undetectable.

The efficacy of a method of treatment described herein may be assessed in comparison to an untreated subject having a comparable diagnosis or to a subject having a comparable diagnosis who is receiving standard of care therapy. In some embodiments, the efficacy of a method described herein is compared to the subjected treated in accordance with a method described herein prior to the first administration.

### Combination Therapies

In some embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokine antagonists (for example, anti-TNF agents such as infliximab, adalimumab, golimumab, natalizumab, anti-IL-6 such as tocilizumab and sarilizumab, anti-12/23 such as ustekinumab), cytokines (for example, interleukin-10 or transforming growth factor-beta), anti-trafficking agents (for example, anti-integrins such as vedolizumab and S1P inhibitors such as ozanimod, etrasimod, and fingolomid), hormones (for example, estrogen), or a vaccine. In addition, immunosuppressive or tolerogenic agents including but not limited to anti-thymocyte globulin, calcineurin inhibitors (e.g., cyclosporin and tacrolimus); mTOR inhibitors (e.g., rapamycin, sirolimus); mycophenolate mofetil, antibodies (e.g., recognizing CD3, CD4, CD40, CD154, CD45, IVIG, or B cells); chemotherapeutic agents (e.g., methotrexate, treosulfan, busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

### Dosage Regimens

In one embodiment, the immune effector cells (e.g., T cells) are modified by engineering/introducing chimeric antigen receptors and at least one granzyme gene into said immune effector cells and then infused into a subject. In some embodiments, immune effector cells are modified by engineering/introducing a chimeric receptor and at least one granzyme gene into the immune effector cells and then infused within about 0 days, within about 1 day, within about 2 days, within about 3 days, within about 4 days, within about 5 days, within about 6 days or within about 7 days into a subject.

In some embodiments, an amount of modified effector cells is administered to a subject in need thereof and the amount is determined based on the efficacy and the potential of inducing a cytokine-associated toxicity. In another embodiment, the modified effector cells are CAR+ and CD56+ cells. In some embodiments, an amount of modified effector cells comprises about 10⁴ to about 10⁹ modified effector cells/kg. In some cases, an amount of modified effector cells comprises about 10⁴ to about 10⁵ modified effector cells/kg. In some cases, an amount of modified effector cells comprises about 10⁵ to about 10⁶ modified effector cells/kg. In some cases, an amount of modified effector cells comprises about 10⁶ to about 10⁷ modified effector cells/kg. In some cases, an amount of modified effector cells comprises about 10⁷ to about 10⁸ modified effector cells/kg. In some cases, an amount of modified effector cells comprises about 10⁸ to about 10⁹ modified effector cells/kg. In some cases, am amount of modified effector cells comprises about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹ modified effector cells/kg.

In one embodiment, the modified immune effector cells are targeted to the cancer cells via regional delivery directly to the tumor tissue. For example, in ovarian or renal cancer, the modified immune effector cells can be delivered intraperitoneally (IP) to the abdomen or peritoneal cavity. Such IP delivery can be performed via a port or pre-existing port placed for delivery of chemotherapy drugs. Other methods of regional delivery of modified immune effector cells can include catheter infusion into resection cavity, ultrasound guided intra-tumoral injection, hepatic artery infusion or intrapleural delivery.

In one embodiment, a subject in need thereof, can begin therapy with a first dose of modified immune effector cells delivered via IV followed by a second dose of modified immune effector cells delivered via IV. In one embodiment, a subject in need thereof, can begin therapy with a first dose of modified immune effector cells delivered via IP followed by a second dose of modified immune effector cells delivered via IV. In a further embodiment, the second dose of modified immune effector cells can be followed by subsequent doses which can be delivered via IV or IP. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In some embodiments, the duration between the first and second or further subsequent dose can be about: 0, 1, 2, 3, 4, 5, 6, 7, 8, 9 or 10 years.

In another embodiment, a catheter can be placed at the tumor or metastasis site for further administration of 1, 2, 3, 4, 5, 6, 7, 8, 9, 10 doses of modified immune effector cells. In some cases, doses of modified effector cells can comprise about 10² to about 10⁹ modified effector cells/kg. In cases where toxicity is observed, doses of modified effector cells can comprise about 10² to about 10⁵ modified effector cells/kg. In some cases, doses of modified effector cells can start at about 10² modified effector cells/kg and subsequent doses can be increased to about: 10⁴, 10⁵, 10⁶, 10⁷, 10⁸ or 10⁹ modified effector cells/kg.

### Articles of Manufacture or Kits

An article of manufacture or a kit comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent. Suitable containers for the one or more agent include, for example, bottles, vials, bags and syringes.

### Examples

### Example 1. Granzyme F Production By CD8 T Cells In The Tumor Microenvironment

Granzymes are a class of cytotoxic proteases and are the primary mechanism utilized by T cells to directly eliminate cancer cells. Each granzyme acts upon a unique set of substrates in target cells to induce cytotoxicity through a range of different mechanisms. Granzymes are some of the most differentially regulated genes in CD8+ tumor infiltrating lymphocytes, relative to T cells outside of the tumor microenvironment (TME).

Microarray data from Gene Expression Omnibus (GEO) was examined for expression of granzyme encoding genes in CD8 TIL relative to CD8 T cells from either matched spleens or lymph nodes. T cells were analyzed from the CT26 tumor (n=3), the MC38 tumor (n=2) and the B16 tumor (n=3). Microarray data were analyzed using the Geo2r software, and only significantly altered genes were shown as determined by a corrected P-value<0.05. Quantitative PCR (qPCR) was performed for multiple granzymes genes, Prf1 and Fasl expression in CD8 TIL and CD8 T cells from the spleen. For qPCR data, a one sample t test was used to compare the fold change for each gene to no change in expression. For all genes with a P-value<0.05, a single star indicates significance.

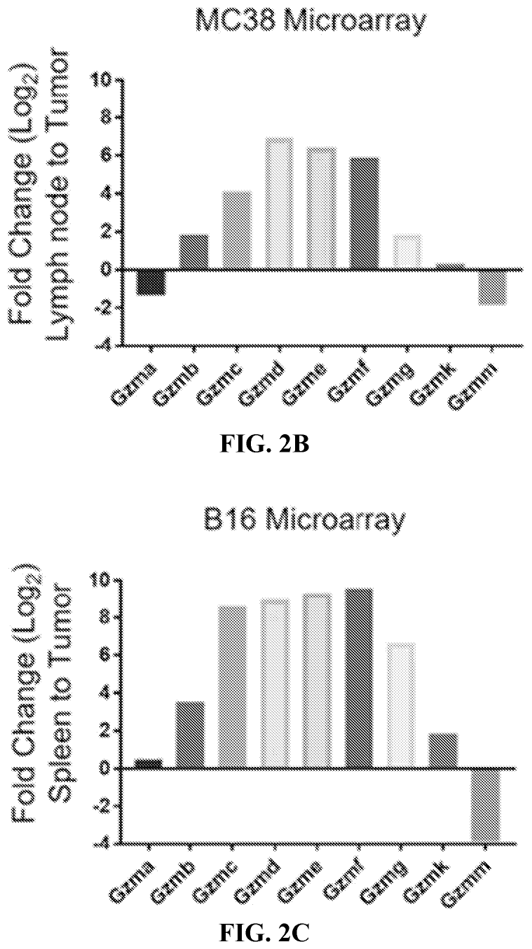

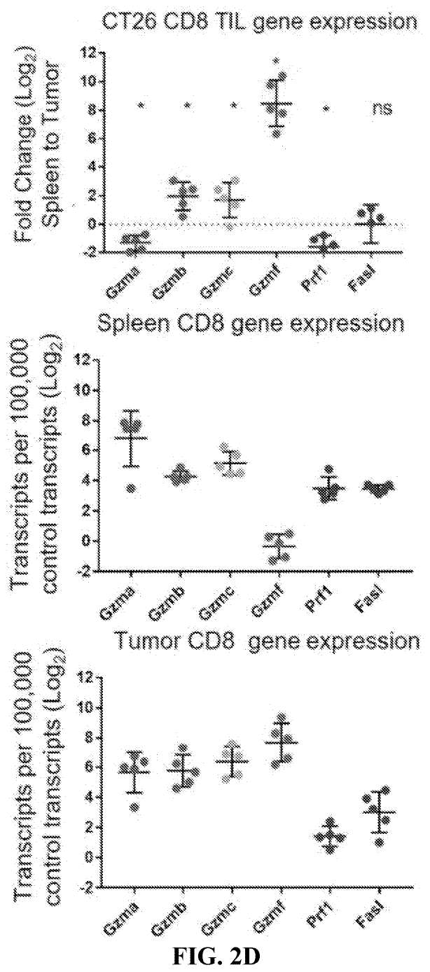

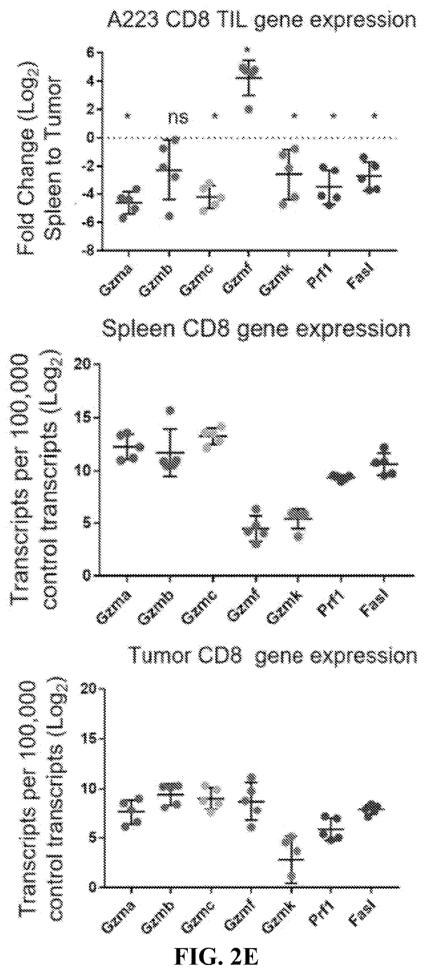

While multiple other granzymes had increased expression in the TME, many of which were members of the Gzmb locus, Gzmf was the most highly upregulated. To determine if this expression pattern is generalizable, publicly available microarray data was used to further examined granzyme expression in CD8 T cells isolated from the MC38 murine colon carcinoma model (Arina et al. Nature Communications 2019; 10 (1): 3959) (FIG. 2B) and the B16 murine melanoma model (Zhou et al., Nature 2014; 506 (7486): 52-7) (FIG. 2C). These results demonstrated that Gzmf is highly upregulated in CD8 TIL in additional models. These findings were confirmed in the CT26 tumor model (FIG. 2) and the head and neck squamous cell carcinoma A223 (FIG. 2E) (Woolaver, et al. Cancer 2021; 9 (1): e001615; White et al., The Journal of clinical investigation 2013; 123 (10): 4390-404) relative to matched splenic CD8 T cells by quantitative PCR (qPCR). In all these tumor models Gzmf was highly upregulated in CD8 TIL relative to CD8 T cells outside the TME suggesting that Gzmf expression is modulated when T cells enter the TME. Given that the expression of Gzmf is highly controlled, it may be a marker, or key mediator, of a specific CD8 T cell phenotype and/or function.

### Example 2: CD3/CD28 Stimulation has a Diverse Influence on Granzyme Expression

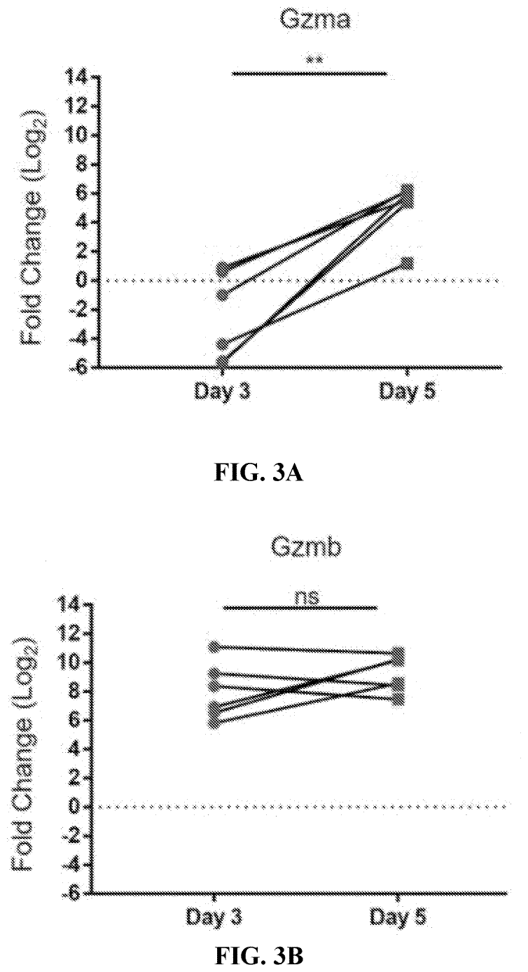

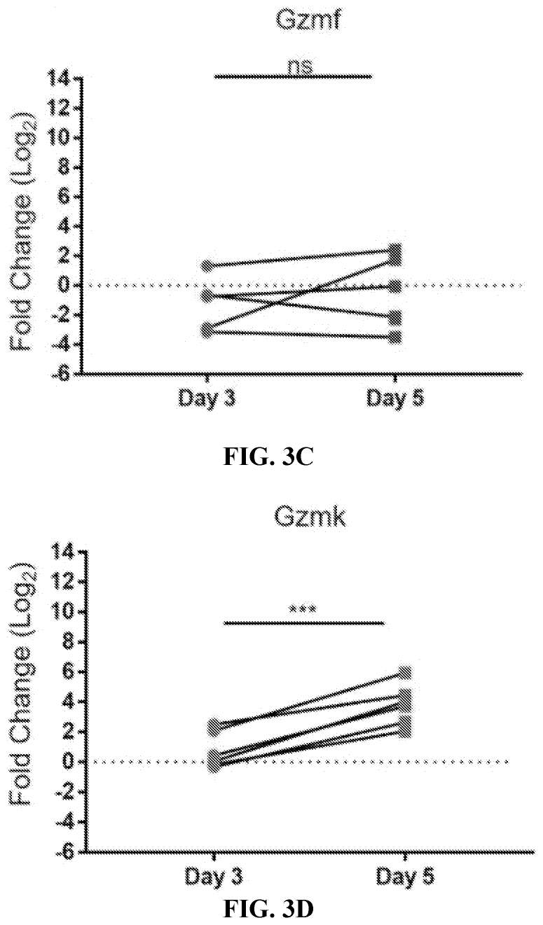

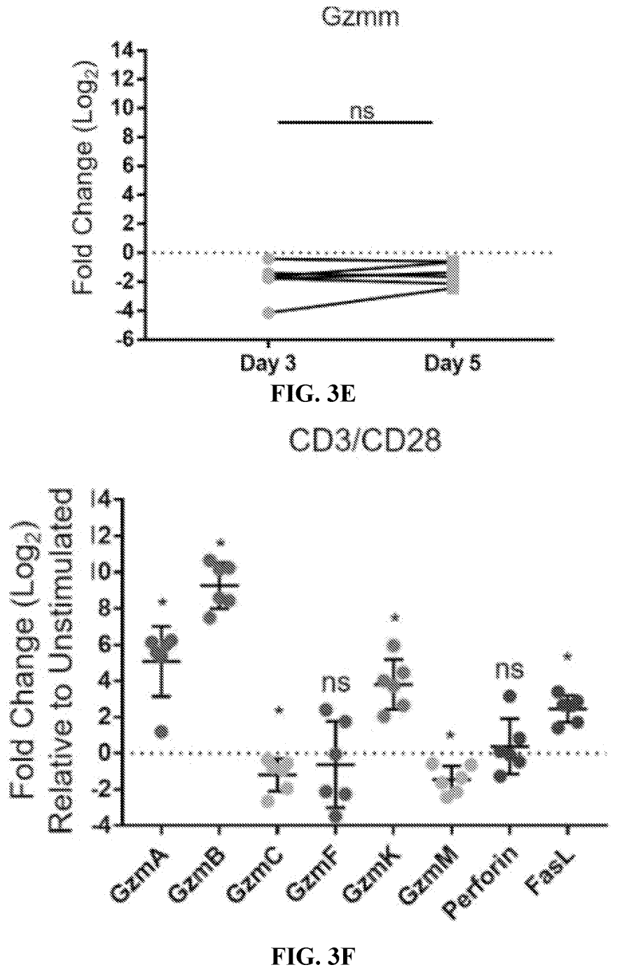

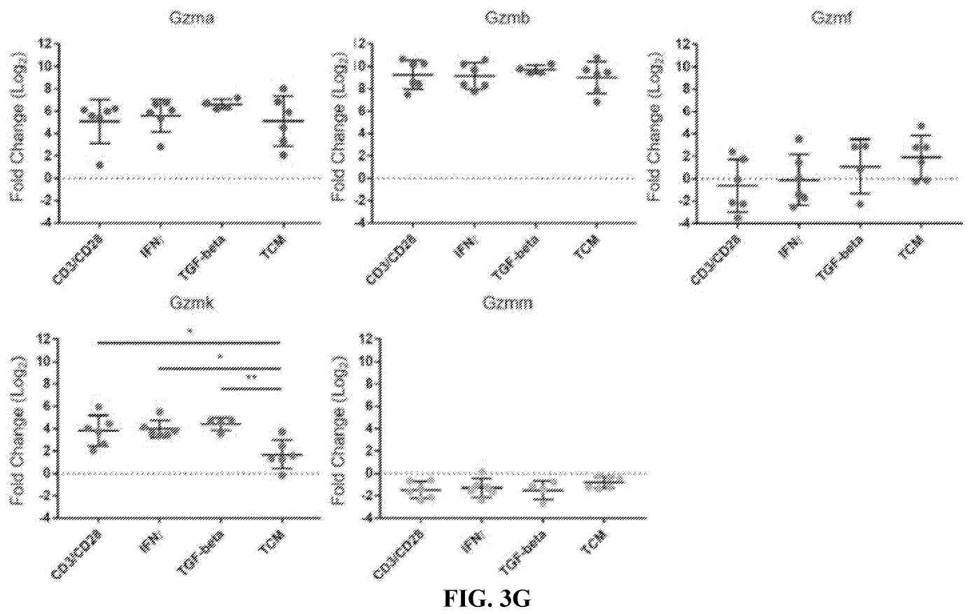

Example 2: CD3/CD28 stimulation has a diverse influence on granzyme expression To elucidate pathways that influence granzyme expression, BALB/c spleens were harvested, isolated CD8 T cells, stimulated these cells in vitro, and determined granzyme expression by qPCR. Relative to unstimulated CD8 T cells, anti-CD3 and anti-CD28 (CD3/CD28) stimulation induced a significant increase in expression of Gzma by Day 5 (FIG. 3A). The expression of Gzmb was high by Day 3 and stayed high until Day 5 (FIG. 3B). These results indicate that Gzma and Gzmb expression are induced through CD3/CD28 stimulation in vitro, but that the rate of Gzma upregulation is slower than the rate of Gzmb. Gzmf, Gzmk, Gzmm (FIGS. 3C-3E) were evaluated in the same manner. Neither the expression of Gzmf nor Gzmm was significantly altered by CD3/CD28; though Gzmk expression increased by Day 5 like Gzma, which is on the same locus. While TCR engagement was sufficient to induce some granzymes, others may require additional or alternative stimuli. These data highlight that expression of granzyme is dynamic and regulated.

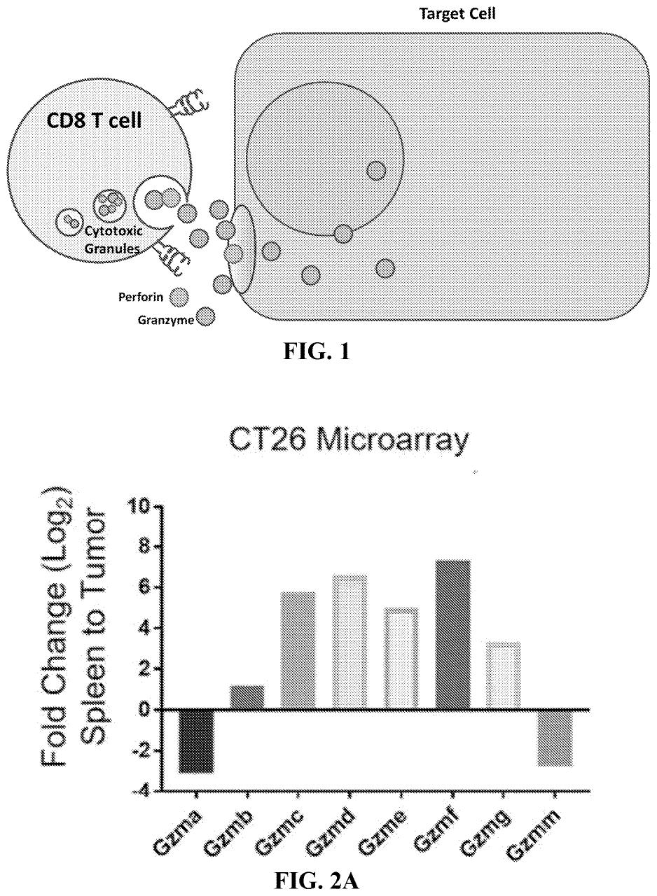

Expression of multiple granzymes, perforin and FasL, was compared 5 days post CD3/CD28 stimulation (FIG. 3F) and Gzmb was the most significantly upregulated gene of all examined. This analysis, in combination with the data shown in FIGS. 1D, 1E and 2C indicates that the expression of Gzmf was low outside the TME.

Other in vitro culture conditions were tested to determine if alternative T cell stimulation induced Gzmf expression. Thus, Gzma, Gzmb, Gzmf, Gzmk and Gzmm expression were examined by qPCR after CD3/CD28 stimulation and three different treatments. Interferongamma (IFNγ) treatment because expression of multiple interferon-inducible transmembrane genes was found to be correlated with Gzmf expression. TGF-beta treatment was tested because TIL expression patterns reflect an environment of strong TGF-beta signaling (Waugh et al., Journal of Immunology (Baltimore, Md: 1950) 2016; 197 (4): 1477-88) and the Gzmf promoter has seventeen G/C-rich sites predicted to bind the Sp1/KLF-like family of transcription factors some of which are known to be regulated by TGF-beta (Ellenrieder, Anticancer Research 2008; 28 (3a): 1531-9).

Finally, the effects of tumor-conditioned medium (TCM) on granzyme expression was tested to determine if soluble factors from the tumor may be controlling Gzmf expression (FIG. 3G). None of these conditions had a significant impact on Gzmf expression relative to unstimulated cells or to CD3/CD28 stimulation alone. However, Gzmk expression was significantly reduced when treated with TCM relative to the other stimulation conditions, suggesting that Gzmk expression was impaired by a soluble factor produced by tumor cells.

Genes correlated with GzmF expression were identified using single cell RNA sequencing of CD8 TIL from the CT26 tumor model. Pearson's R value was determined for the relationship of Gzm F expression with every other significantly expressed gene. The genes with the highest significant R-value are displayed in Table 1. Benjamini-Hochberg (BH) corrected P-values were calculated to consider the false discovery rate.

TABLE 1
Genes correlated with GzmF expression

| Gene | R value | P-value | BH |
|---|---|---|---|
| Irf8 | 0.185203 | 1.06E−55 | 8.85E−53 |
| Ifitm3 | 0.189345 | 3.50E−58 | 3.14E−55 |
| Prf1 | 0.193603 | 8.53E−61 | 8.29E−58 |
| Gm44040 | 0.204955 | 4.56E−68 | 4.84E−65 |
| Lgals3 | 0.225798 | 1.36E−82 | 1.59E−79 |
| Serpinb9b | 0.244256 | 9.43E−97 | 1.22E−93 |
| Spp1 | 0.267869 | >1.0E−100 | >1.0E−100 |
| Gzmb | 0.275442 | >1.0E−100 | >1.0E−100 |
| Ifitm1 | 0.295241 | >1.0E−100 | >1.0E−100 |
| Ifitm2 | 0.333623 | >1.0E−100 | >1.0E−100 |
| Gzme | 0.348327 | >1.0E−100 | >1.0E−100 |
| Gzmd | 0.377166 | >1.0E−100 | >1.0E−100 |
| Gzmc | 0.487932 | >1.0E−100 | >1.0E−100 |
| Gzmf | 1 | >1.0E−100 | >1.0E−100 |

### Example 3: Gzmf Expression is Increased in Exhausted CD8 TIL

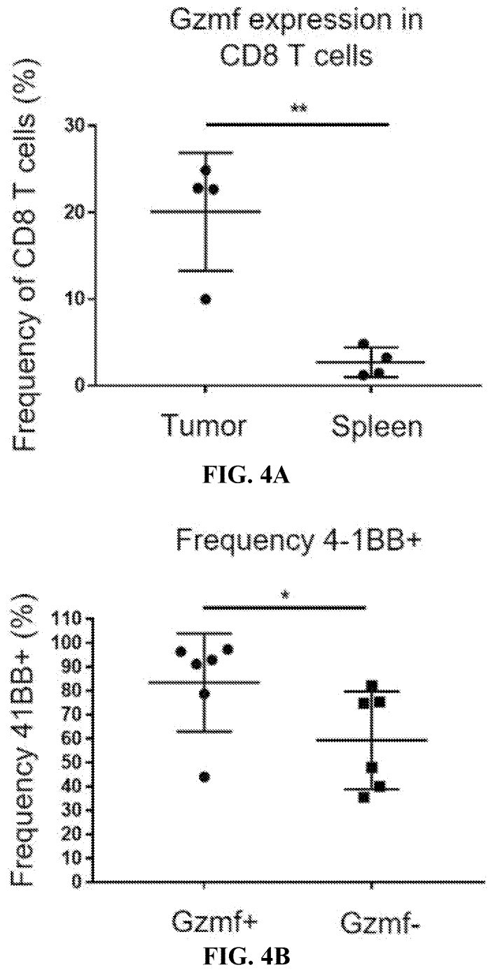

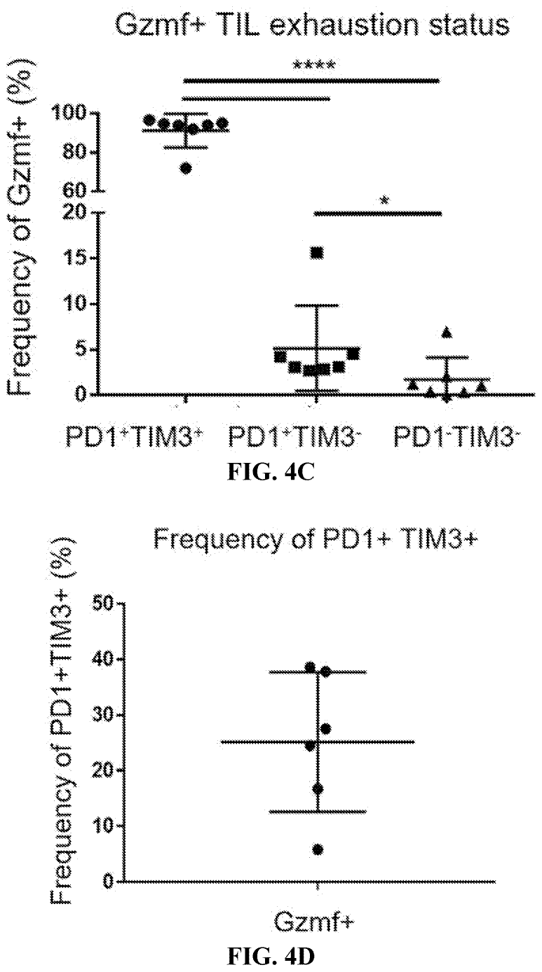

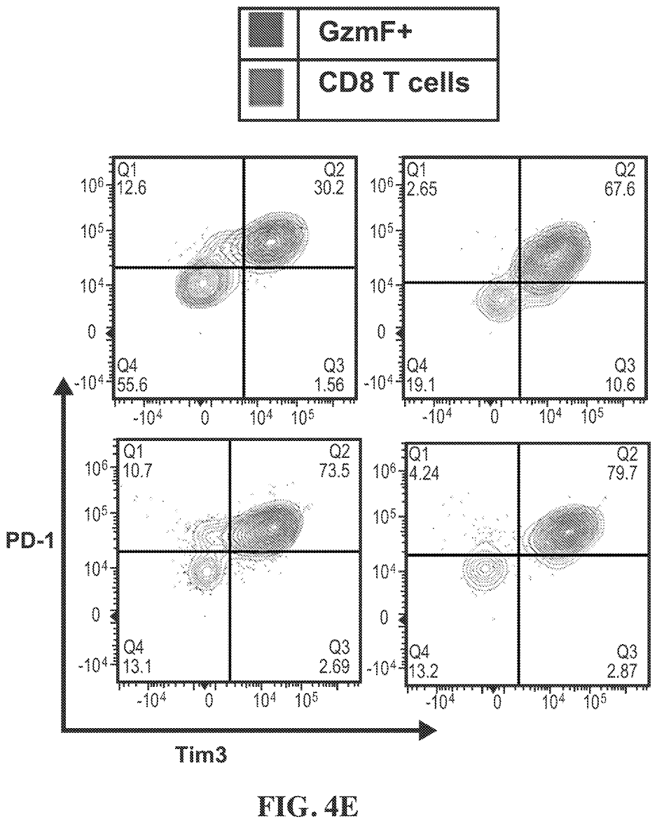

Using a flow cytometry-based technique for the detection of RNA transcripts (PrimeFlow), CD8 TIL were interrogated on a single-cell level for the expression of Gzmf transcripts and cell surface proteins simultaneously. As expected, detection of Gzmf by PrimeFlow on the single cell level and qPCR-based on bulk CD8 TIL showed increased expression in TIL relative to splenic CD8s (FIG. 4A). In addition, Gzmf expressing cells were predominantly 4-1BB positive, suggesting antigen experience (FIG. 4B). Gzmf expressing cells were also positive for the inhibitory receptors PD1 and TIM3 (FIG. 4C), suggesting that Gzmf expression is induced in exhausted CD8 T cells (Sakuishi et al., J Exp Med 2010; 207 (10): 2187-94). However, only a subpopulation of PD1 and TIM3 expressing cells express Gzmf (FIG. 4D). Representative flow plots of inhibitory receptors suggested that Gzmf is only expressed in a subpopulation of exhausted CD8 TIL (FIG. 4E).

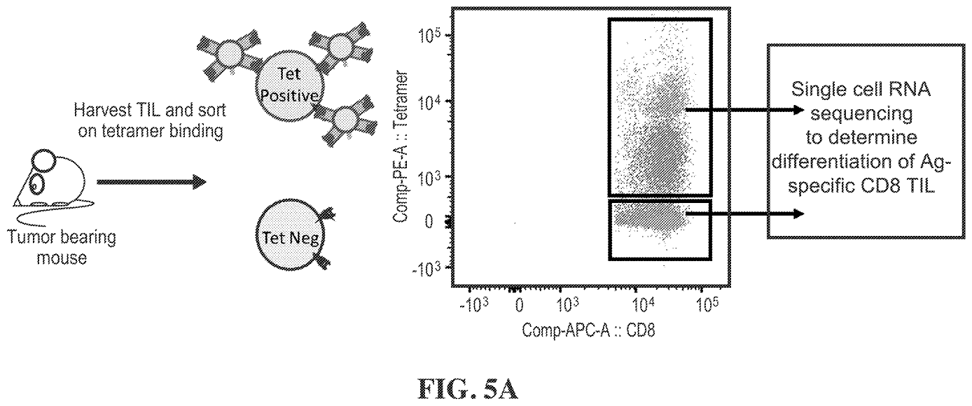

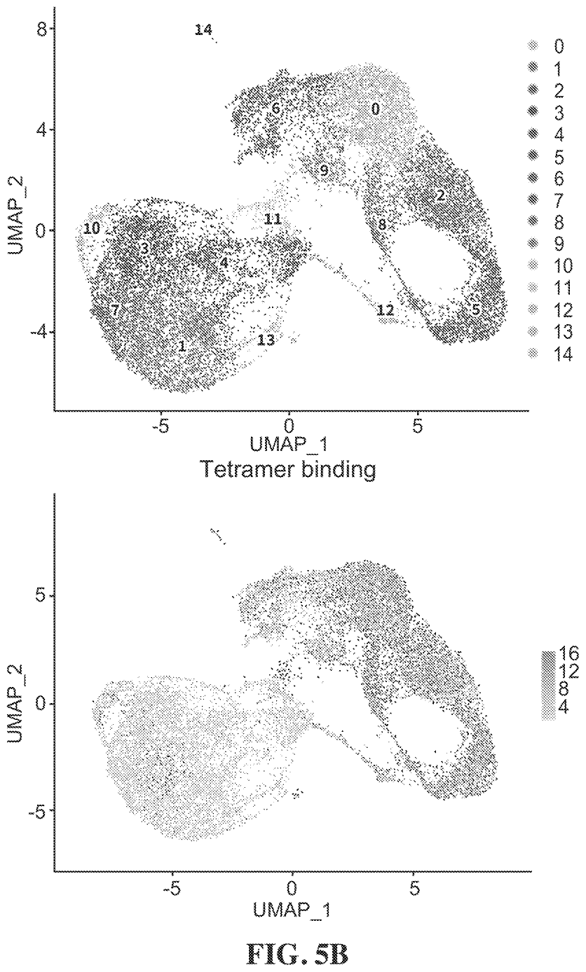

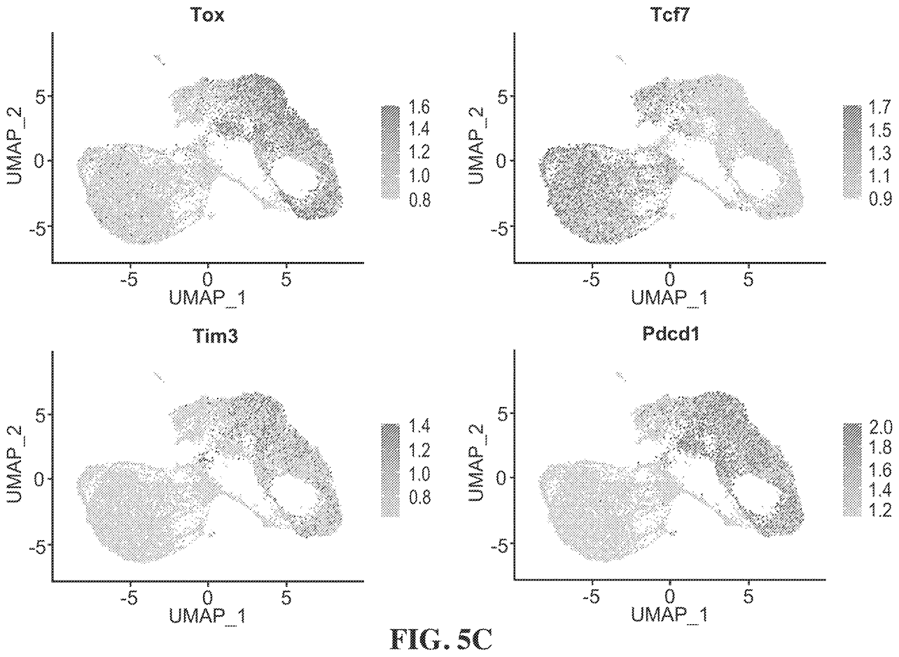

FIG. 5 shows an exemplary method using single cell RNA sequencing of CD8 TIL indicates granzyme F-high expressing cells are unique from both granzyme A and B expressing cells, and that it is therefore likely these TIL utilize a unique mechanism of cytotoxicity in their elimination of cancer cells.

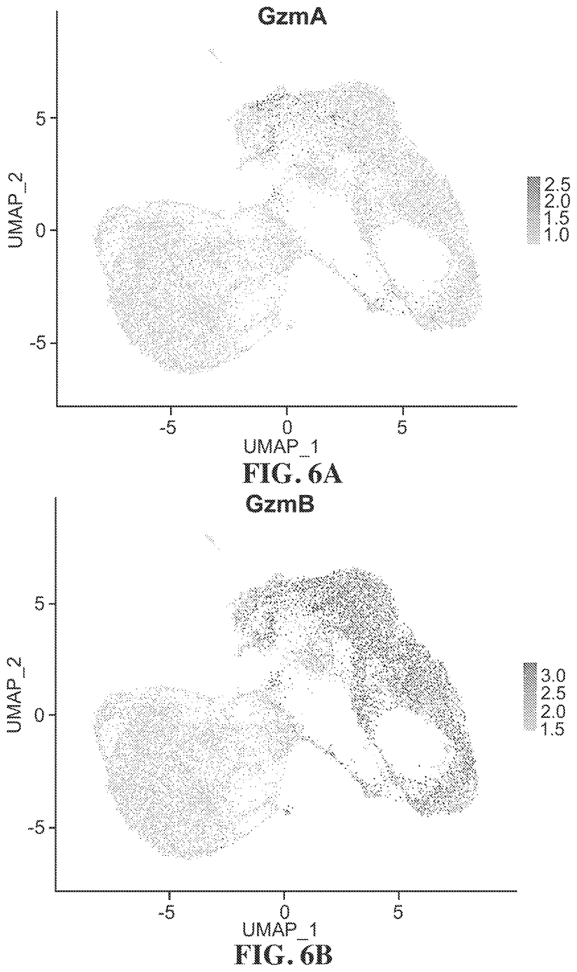

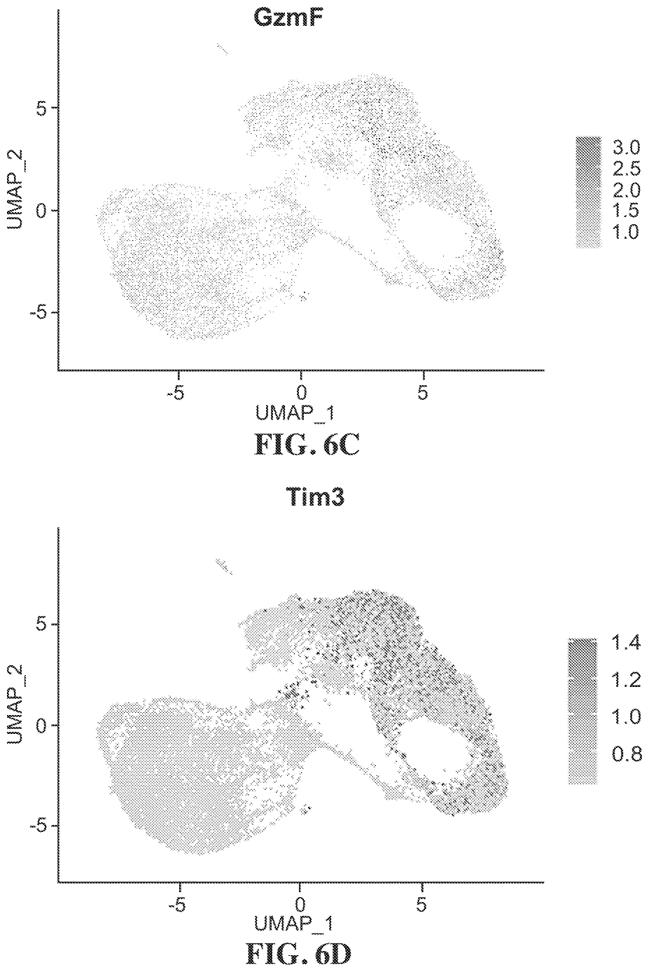

FIGS. 6A-6D show that GzmA expression is restricted to more progenitor-like clusters while GzmF is specifically expressed in more differentiated clusters.

### Example 4: Construction of T Cells Overproducing Granzymes

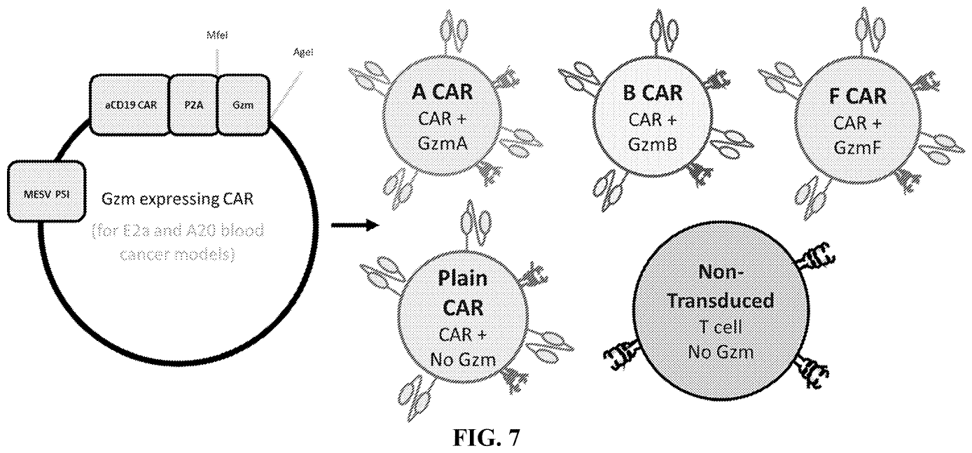

Recombinant granzyme F has previously been shown to induce a unique form of cell death, characterized as being caspase-independent and resulting in rupture of target cell plasma membrane. FIG. 7 is a schematic depicting an exemplary method described herein for the construction of T cells expressing a chimeric antigen receptor (CAR) and overproducing at least a single granzyme. As shown in FIG. 7, individual plasmids were constructed to contain an anti-CD19 CAR, a P2A linker and a single granzyme encoding gene, under the control of a MSCV promoter. Three plasmids were constructed to each individually express either granzyme A (A CAR), granzyme B (B CAR), granzyme F (F CAR) The plasmids also contained a retroviral psi packing element (MESV Psi). A control plasmid encoding an anti-CD19 CAR with no granzyme gene (plain CAR) was also constructed. As a control for experiments, a non-transduced T cell containing no plasmid was included. Overproduction of granzyme F was performed to determine if the mechanism of cell death is leverageable to improve the cytotoxic capacity of TIL over producing granzyme F, and whether the induction of different forms of T cell-mediated cytotoxicity can modulate the immunogenicity of the TME. These experiments are designed to provide insight into how to improve adoptive T cell therapies by directly improving cytotoxicity, the terminal step of T cell interaction with tumor cells.

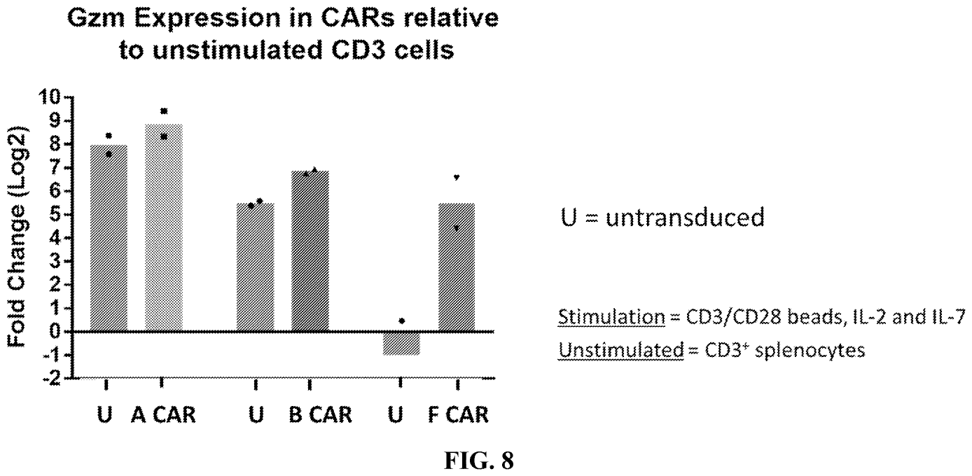

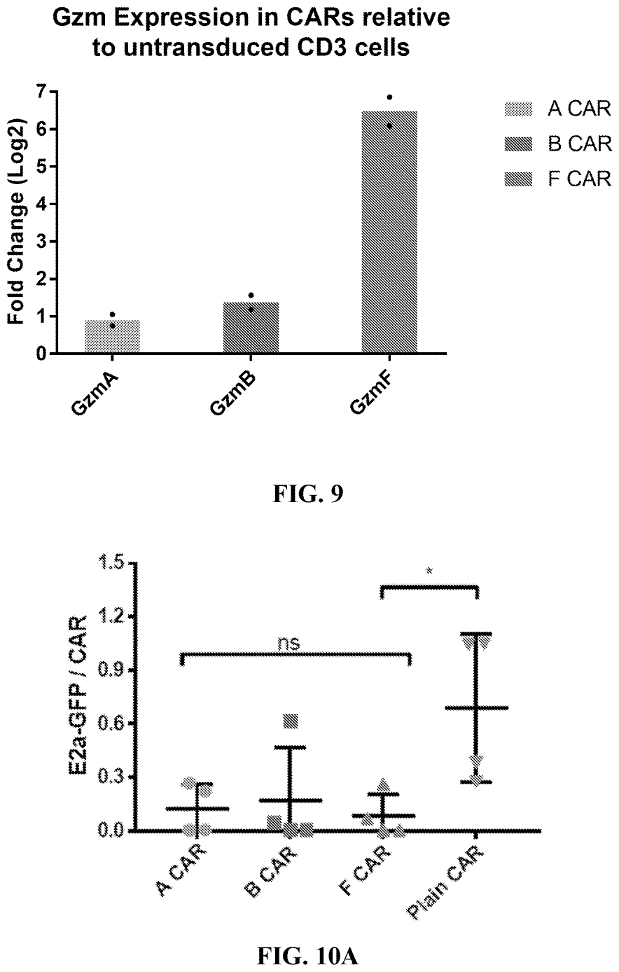

Plasmids were introduced into T cells to generate CAR T cells overproducing granzymes. FIG. 8 shows Gzm expression in stimulated untransduced and CAR T cells overproducing granzymes relative unstimulated CD3+ splenocytes. Stimulated cells were cultured for 2 days with anti-CD3/CD28 bead-bound antibodies, IL-2 and IL-7. Results are shown relative to sample matched unstimulated CD3+ splenocytes as a Log 2 fold change based on qPCR measurements. T tests were performed to determine if each gene had a significant fold change between experimental and control groups. FIG. 9 shows Gzm expression in CAR T cells relative to untransduced CD3 cells. Results are shown as a Log 2 fold change based on qPCR measurements.

### Example 5: Granzyme Producing T Cells Improves T Cell Cytotoxicity

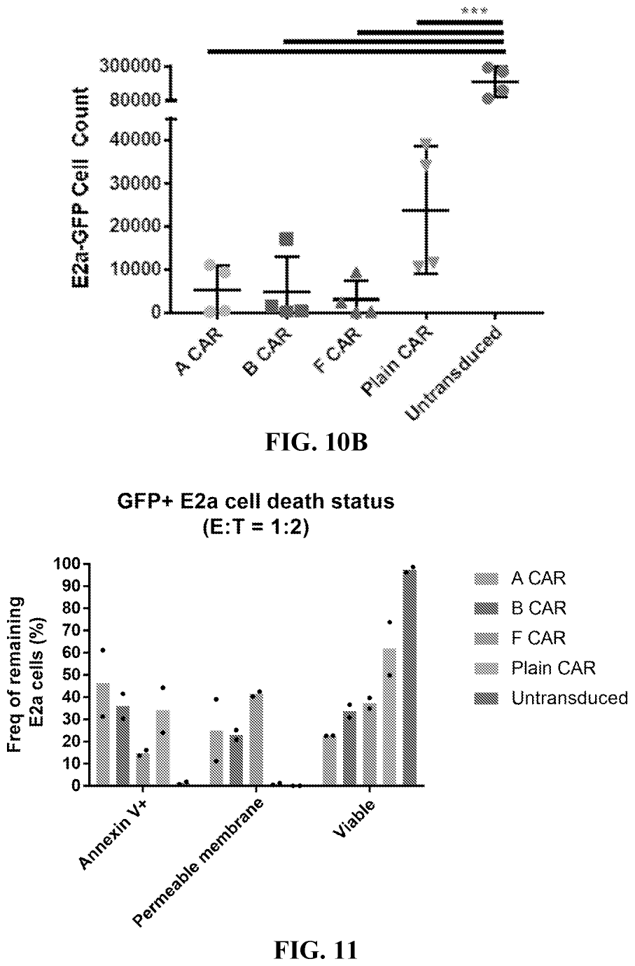

CARs or untransduced T cells were cultured overnight with E2a cancer cells expressing GFP (E2a-GFP). A, B or F CAR groups are Gzm overexpressing and the Plain CAR group are CAR cells that do not overexpress any Gzm. Equivalent number of E2a-GFP cells were seeded in each well and all CAR groups were normalized so the final concentration of CAR+ cells was at a 1:2 effector to target (E:T) ratio. Untransduced T cells were plated at an equivalent cell density as the highest total T cell density of any of the CAR groups. FIG. 10A shows the ratio of total remaining E2a-GFP cells after the coculture to remaining viable CAR+ cells. The Gzm F overexpressing cells have improved cytotoxic activity over the Plain CAR. FIG. 10B shows the E2a-GFP cell counts. All of the CARs decrease the total E2a-GFP cell count. These data indicate that Gzm overexpressing CARs have improved cytotoxicity relative to plain CARs and untransduced T cells.

### Example 6: Granzyme Production in T Cells Alters Form of Cell Death Induced by T Cells

To further examine function of Gzms F, A and B, CAR T cells overexpressing these Gzms, along with plain CAR and untransduced T cells were cocultured with E2a-GFP cells, a model for acute lymphoblastic leukemia. The percent of E2a-GFP cells that were viable after CAR coculture was determined by a cell permeable dye and annexin V staining and is presented as “Frequency of remaining E2a cells”. The persisting cancer cells were examined for the permeability of their cell membrane and the externalization of phosphatidylserine by annexin V staining, a marker of apoptotic cell death when the cell membrane is intact. Gzm F overexpressing CAR T cells induced fewer apoptotic cells suggesting a more immunogenic cell death, as shown in FIG. 11. The frequency of annexin V single positive E2a-GFP cells or E2a-GFP cells with a permeable membrane was determined as a percentage of total non-viable E2a-GFP cells and indicates that coculture with Gzm F over expressing CAR T cells induce fewer E2a-GFP cells to undergo apoptotic cell death relative to other coculture groups.

### Example 7: CAR T Cells that Overexpress Gzmf (F CARs) have Increased Cytotoxic Activity and Altered Cell Death Induction

To further understand the function of Gzmf, three granzyme overexpressing anti-CD19 CAR T cells were constructed: Gzma overexpressing (A CAR), Gzmb overexpressing (B CAR) and Gzmf overexpressing (F CAR). The granzyme genes were inserted downstream of the CAR sequence and attached by a P2a linker. Additionally, the following controls were used: a CAR that did not have overexpression of any granzyme genes (Plain CAR) and T cells that were cultured and stimulated the same way but were not genetically engineered (Untransduced). Granzyme expression was not deleted in any construct so all CAR models retained endogenous expression of all granzymes.

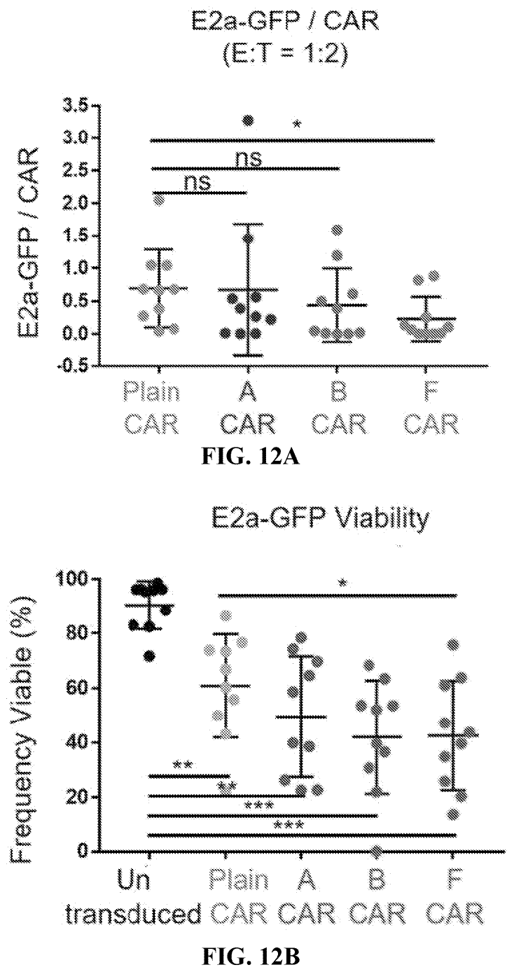

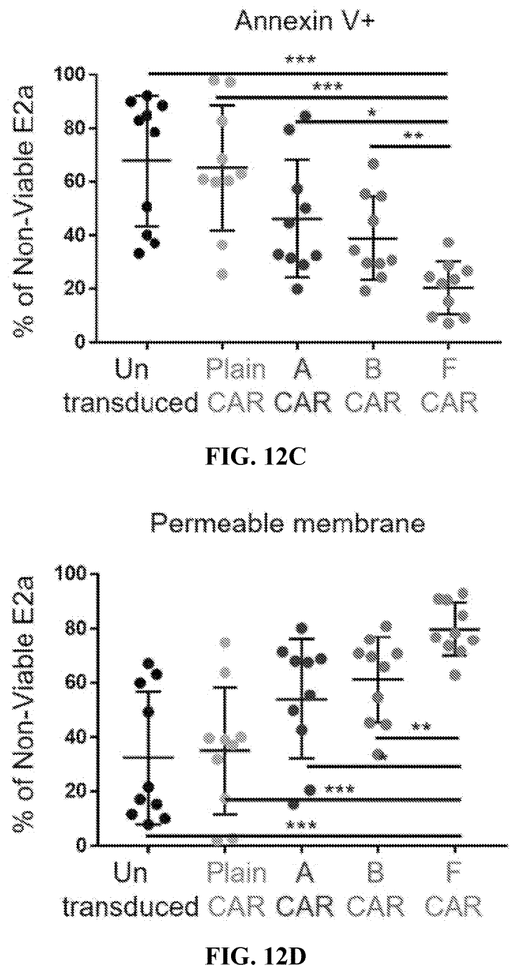

To determine the effect granzyme overexpression has on cytotoxicity, CAR T cells were co-cultured overnight with the CD19 expressing ALL cell line E2a that expresses green fluorescent protein (GFP). The frequency of E2a-GFP cells was normalized to CAR T cells as determined by flow cytometry after coculture. The greatest decrease in E2a-GFP cells was observed in the F CAR samples (FIG. 12). The frequency of cells that remained in the co-culture that were still viable was also determined and again F CARs had a significant reduction of viable E2a-GFP cells relative to Plain CAR samples, and all groups had a reduction in the frequency of viable E2a-GFP cells relative to the untransduced treated group (FIG. 12B). While F CAR cells were significantly more cytotoxic than Plain CARs, there were no significant differences in anti-cancer cytotoxicity observed between the various granzyme overexpressing groups.

The F CARs had reduced apoptotic cells, as determined by Annexin V staining, (FIG. 12C) and an enrichment of cells with a permeable membrane (FIG. 12D) relative to the other groups. The unique cytotoxic activity of Gzmf, inducing membrane permeabilization rather than externalization of phosphotidyl serine, could have downstream effects in vivo. Increasing cell membrane rupture is associated with increased release of damage associated molecular patterns (DAMPs) and subsequent invigoration of the immune response (Chen et al., Nature Reviews Immunology 2010; 10 (12): 826-37; Vénéreau et al., Front Immunol 2015; 6:422).

These results suggest that Gzmf overexpression induces an alternative form of cytotoxicity, and that overexpression alone is sufficient to alter the form of cell death induced in target cells.

### Example 8: F CARs have Decreased Viability Compared to Other CAR Groups and Gzm Overexpressing CARs have Decreased Expression of CD69

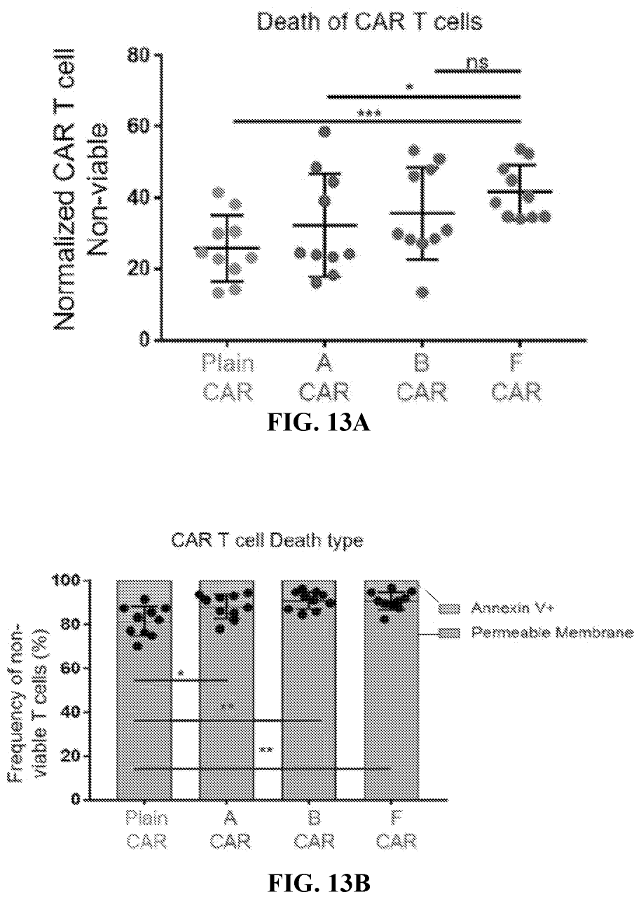

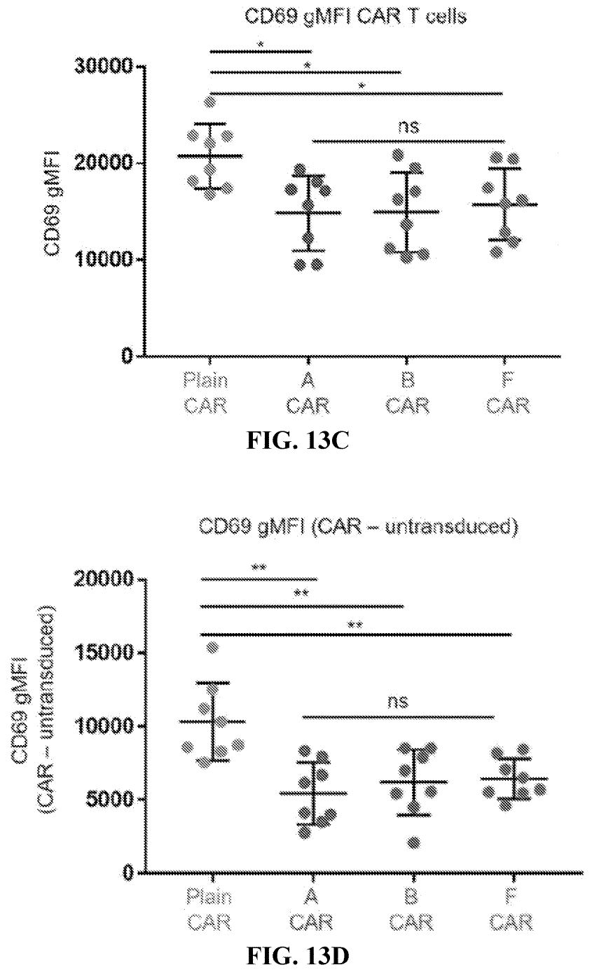

To investigate the cell intrinsic effect granzyme expression has on the survival of T cells, granzyme overexpressing CAR T cells were examined after co-culture with E2a-GFP cells overnight. F CARs had the most cell death and had significantly more cell death relative to the Plain CARs and the A CARs, suggesting that Gzmf expression is detrimental to the T cell it is produced in (FIG. 13A). CAR T cell death was normalized in each sample by taking the percentage of non-viable CAR+ T cells and subtracting the percentage of non-viable CARnegative T cells, to correct for cell death that occurred in samples independent of granzyme overexpression. This data suggest that CAR T cell death was a cell intrinsic activity since CAR-negative cells had less cell death than CAR-positive cells; strongly suggesting that this was not caused by off target toxicity acting on total T cells in culture.

The form of cell death cultured T cells were undergoing by measuring permeable cell membranes or expression of annexin V alone as a percentage of total non-viable cells in CAR T cells (FIG. 13B). While all dying CAR T cells had significant bias towards a permeable membrane rather than annexin V staining, dying granzyme overexpressing CAR T cells were enriched for permeabilized cell membranes over CAR T cells that did not over express granzymes. This bias towards membrane permeabilization may be indicative of either the type of cell death occurring or the rate of that cell death, given that apoptotic cells will eventually lose membrane integrity in vitro. The decrease in frequency of annexin V single positive cells in granzyme overexpressing groups reflects a physiological change in T cell viability when they have increased expression of granzymes.

Given the improved cytotoxicity against cancer cells but reduced viability of granzyme overexpressing CAR T cells, it was determined if granzyme overexpression impacted the activation status of CAR T cells. The activation status of T cells was examined after being cocultured with E2a-GFP cells and it was found that Plain CAR T cells had significantly higher geometric mean fluorescent intensity (gMFI) for CD69 expression (FIG. 13C), and that this is consistent after normalizing to CD69 gMFI expression to CAR-negative T cells in the same culture to correct for CD69 expression from non-antigen-specific T cells (FIG. 13D).

Together these results reveal that granzyme overexpression, and particularly Gzmf overexpression, reduces the viability of CARs resulting in reduced activation of CAR T cells. Notably, all samples had increased CD69 expression on CAR T cells relative to other T cells in the same culture (FIG. 13D), consistent with changes in CD69 expression resulting from antigen-specific interactions. Reduced CD69 expression and viability of granzyme overexpressing T cells suggests that granzyme overexpression may lead to the elimination of activated cells. Without wishing to be bound by theory, these results may suggest a mechanism by which exhausted TIL eliminate themselves through granzyme expression. Gzmf consistently has more cell intrinsic cytotoxic effects than other granzymes in this system.

### Example 9: F CARs have Impaired Control of Cancer Growth in Vivo while B CARs and Plain CARs have Comparable Efficiency

Anti-CD19 CAR T cells were transferred into leukemia bearing mice to determine the impact granzyme expression has on control of target cell growth and CAR persistence in vivo.

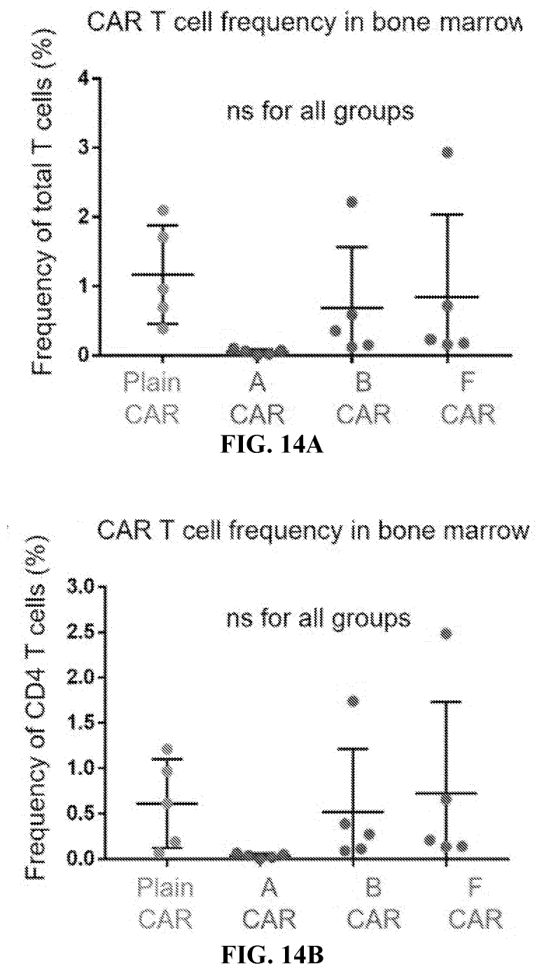

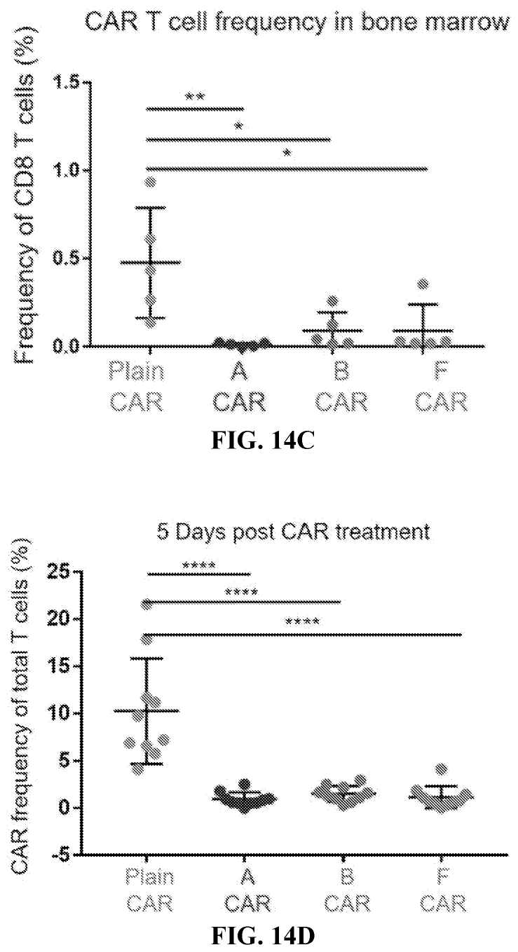

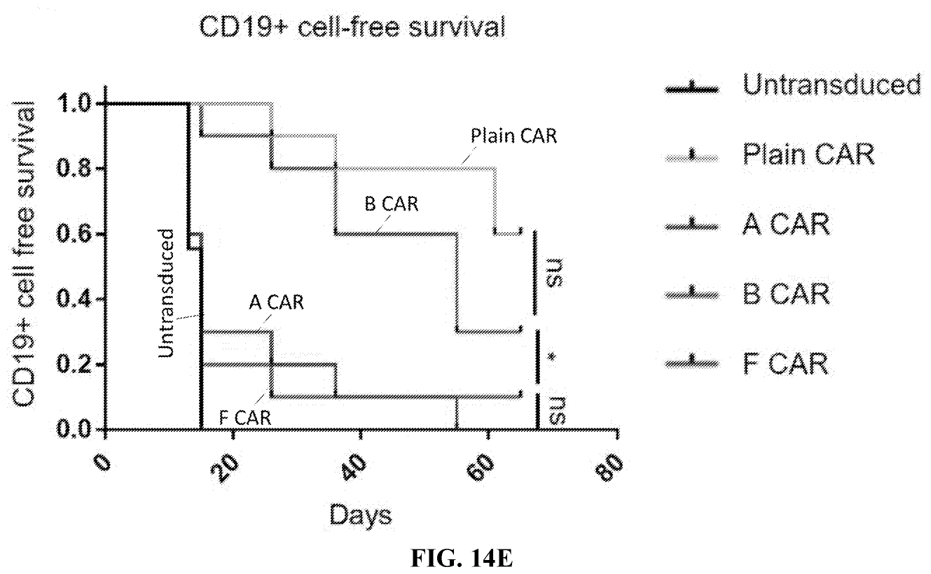

CAR persistence was measured in the bone marrow, where these leukemic cells concentrate, 55 days post-adoptive CAR T cell transfer as a fraction of total T cells (FIG. 14A), CD4 T cells (FIG. 14B), and CD8 T cells (FIG. 14C). There was no difference in CAR T cell persistence between CARs as a percentage of bulk T cells or as a percentage of CD4 T cells, but there were more Plains CARs as a percentage of CD8 T cells relative to granzyme overexpressing CAR groups. The frequency of CD8 CAR T cells was less than CD4 CAR T cell in granzyme overexpressing samples. A CAR cells seemed to have the worst persistence in the bone marrow at the timepoint examined across all T cell subsets. While this was not significantly different than B CAR and F CAR results, it is worth noting that Gzma is located on a different locus and has been shown to have a pro-inflammatory role (Wilson et al., PLOS Pathogens 2017; 13 (2): e1006155; Metkar et al., Immunity 2008; 29 (5): 720-33) which may contribute to this decrease in persistence in the bone marrow.

Plain CARs were comparable in their frequency of CD4 or CD8 cells at this timepoint in the bone marrow, and that all granzyme overexpressing CAR groups were a similar frequency of CD4 cells but were significantly reduced as a percentage of CD8 cells. These data suggest that CD4 CARs were unaffected by granzyme overexpression, indicative of a biological difference between CD4 and CD8 T cells in their susceptibility to granzyme self-directed cytotoxicity. The frequency of CAR T cells as a percentage of T cells in blood was determined 5 days after CAR T cell adoptive transfer (FIG. 14D) and determined that Plain CARs persist more efficiently than all granzyme overexpressing CARs in the blood at this timepoint.

To determine how the granzyme overexpressing CAR T cells would influence target cell growth, the fraction of viable CD19+B220+ lymphocytes in the blood was measured over time (FIG. 14E). Plain CARs and B CARs controlled CD19 expansion the best. The untransduced T cells, the A CARs, and F CARs did not control CD19+ cell expansion. These results suggest that different granzymes have different roles in the anticancer response: granzyme B is well tolerated while expression of granzymes A and F may be toxic to the T cells which produce them. These conclusions are consistent with F CAR impairing its persistence and control of target cell expansion over time, even if in vitro granzyme overexpression increases the cytotoxic activity of CARs against target cells. Given that Gzmf is expressed by more exhausted cells (FIG. 4C) its capacity to eliminate the exhausted cell producing it may not be an artifact of its increased cytotoxic activity but instead a mechanism by which exhausted T cells are actively eliminated.

### Methods for Examples 1-9

### Mice and Cell Lines:

C57BL/6 (B6) mice were purchased from The Jackson Laboratory and BALB/cAnNCr (BALB/c) mice were purchased from Charles River Laboratories. All animal protocols were reviewed and approved by the Institutional Animal Care and Use Committee at the University of Colorado School of Medicine.

E2aPbx (E2a) and E2a-GFP are murine pre-B ALL cell generated by the transduction of the human E2A-PBX (TCF3-PBX1) transgene into the bone marrow of CD3ε^{-/-}C57BL/6 mice. CT26 cells derived from an NMU-treated BALB/c mice is a model for colon carcinoma. They were purchased from ATCC in 2014 and tested for mycoplasma contamination by PCR at the Barbara Davis Center Bioresource Core prior to freezing aliquots, which were used for no more than one month after thawing.

Primary mouse cells and cell lines were cultured in complete medium [RPMI with Lglutamine, 10% FBS, 100 U/ml each penicillin and streptomycin, 1 mmol sodium pyruvate, 10 mmol HEPES, and 1×MEM nonessential amino acids] primary cells were cultured with 0.05 mM 2-mercaptoethanol, and B6 primary cells also received glutaMAX (gibco).

### Tumor Challenge and TIL Processing:

For CT26 tumor studies mice were injected subcutaneously with 1×10⁵ CT26 tumor cells in 100 μL 1×PBS (Life Technologies) in both hind flanks (Slansky et al. Immunity 2000; 13 (4): 529-38 doi 10.1016/s1074-7613). Tumors were harvested after 14 days of tumor growth challenge. TIL and splenocytes were isolated as described (Slansky et al. Immunity 2000; 13 (4): 529-38 doi 10.1016/s1074-7613). Briefly, CT26 tumors were resected from BALB/c mice's rear limbs where they were implanted. Tumors were placed immediately in 5 mL of serum-free RPMI 1640 medium and minced using a razor blade, before treatment for 25 min at 37° C. with 0.1 mg/ml Liberase (Research Grade, Dispase Low) according to the manufacturer's instructions (Roche Life Science). Tumor cells were filtered through a 100-μm cell strainer and washed in complete medium. Splenocytes were resected from BALB/c mice, mechanically dissociated, and filtered through a 100-μm cell strainer, incubated for ~5 min with ammonium chloride-potassium lysis (RBC lysis) buffer (Krusbeek et al., Current Protocols in Immunology 1995; 3), and washed in complete medium. Cells were counted and viability measured prior to downstream applications. All samples were normalized to input an equivalent number of viable cells for downstream experimentation.

A220 tumor samples and matching spleens were prepared similarly to CT26 tumors and as described previously (White et al., The Journal of Clinical Investigation 2013; 123 (10): 4390-404); Woolaver et al. J Immunother Cancer 2021; 9 (1).

### Microarray Data:

Publicly available microarray data was found on GEO. Experiments that examined CD8 T cells from the TME in mice with matching CD8 T cell expression data from outside the TME, were selected for analysis. Using the Geo2R software data were probed for expression patterns of granzyme genes. Data from the CT26 (GSE79858) (Waugh et al., Journal of Immunology (Baltimore, Md: 1950) 2016; 197 (4): 1477-88), the MC38 (GSE111492) (Arina et al. Nature Communications 2019; 10 (1): 3959), and the B16-F10 (GSE53388) (Zhou et al., Nature 2014; 506 (7486): 52-7) tumor models was analyzed.

qPCR:

CD8 T cells were enriched by magnetic negative-selection using Stemcell Technologies EasySep Mouse T cell Isolation Kit (Cat #19851A). RNA was isolated from 1*106 CD8+ T cells using Qiagen's RNeasy Mini-RNA Purification kit according to manufacturer's instructions.

RNA concentration was determined using a Nanodrop Spectrophotometry and cDNA was synthesized using iScript cDNA Synthesis Kit (cat #1708891). cDNA was then used as templates in real time quantitative PCR (qPCR) using SYBR green (Thermo Fisher's Power SYBR Green PCR Master Mix Product #4368706) according to manufacturer's instructions. Primers used for each gene probed are listed in Table 2. Ct values generated from qPCR experimentation were used to determine mRNA expression using delta delta Ct method, where Ct values for a given gene of interest are normalized to Ct values of a control genes that have less variability in their expression. For these experiments Gapdh, Rn18s, and Ubc were used as control genes.

TABLE 2
Primers designed for quantification of granzymes and other genes by qPCR.

| Target | | |
|---|---|---|
| Gene | Forward Primer | Reverse Primer |
| Gzma | AGACCGTATATGGCTCTACT | CCCTCACGTGTATATTCATC |
| Gzmb | ATCAAGGATCAGCAGCCTGA | TGATGTCATTGGAGAATGTCT |
| Gzmc | GGAGATAATCGGAGGCAATGAG | TTCCCACCAACTTTCAGAAACT |
| Gzmd | TGCGAGAGGTTCAACTGGTC | CCCCGAGTCACCCTTGAAA |
| Gzmf | TGAGGTTTGTGAAAGATAATG | TCACTGGTGTTGTCCTTATC |
| Gzmm | ATGGAGGTCTGCTGGTCC | TCCAGGAGCTGTAGGGGG |
| Gzmk | TGTCCGTGCTCCTTAACACC | GCAGCAGTGCGAAGCTTTAT |
| Prf1 | GTACAACTTTAATAGCGACACAGT | AGTCAAGGTGGAGTGGAGGT |
| | A | |
| Fasl | GCAGAAGGAACTGGCAGAAC | TTAAATGGGCCACACTCCTC |
| Gapdh | CATCACTGCCACCCAGAAGACTG | ATGCCAGTGAGCTTCCCGTTCA |
| | | G |
| Rn18s | GGCTACCACATCCAAGGA | GCTGGCACCAGACTTGC |
| Ubc | AGCCCAGTGTTACCACCAAG | ACCCAAGAACAAGCACAAGG |

### In Vitro Stimulation:

To determine what conditions may contribute to differential granzyme expression, in vitro stimulation experiments were performed. Splenocytes were harvested and CD8 T cells magnetically isolated as described above. 1×106 unstimulated cells were taken for direct preparation of RNA as a control. CD8 T cells were resuspended at 1×10⁶ cells/mL and for activation of CD8 T cells 25 μL of anti-CD3/CD28 beads (Gibco reference #11453D) were added per 1×10⁶ cells. All in vitro cultured cells received CD3/CD28 beads for 3 days and 40 U/mL IL-2 for the entire culture. Samples were also treated with either 10 ng/mL IFNγ (purchased from Biolegend), 50% CT26 tumor conditioned media (produced by taking supernatant from CT26 cells that were cultured for 2 days on complete media) or 2 ng/ml TGFbeta (purchased from Biolegend), these treatments were maintained for the duration of in vitro culture. On day 3 of culture 1×10⁶ cells were removed and used to isolate RNA. After 3 days CD3/CD28 beads were removed from samples and cells were resuspended at 1×106 cells again. At day 5 of culture RNA was isolated from 1×10⁶ cells. Isolated RNA from all samples and timepoints was used for qPCR.

### Flow Cytometry and PrimeFlow:

For flow cytometry analyses, 2×10⁶ cells were washed in PBS and stained with a fixable viability dye (Table 3Table 5) for 30 minutes in the dark, per manufacturer's instructions. Samples were then washed with flow buffer [1×PBS, 2% FBS, and 0.1% sodium azide, as described (Slansky et al., Immunity 2000; 13 (4): 529-38)] and stained for surface markers. 50 μL of an antibody cocktail was added to each sample and incubated for 30 minutes. In experiments requiring Annexin V staining samples were surfaced stained, washed with 1× Annexin V staining Buffer, and stained with 5 μL of annexin V per 100 μL of sample for 15 minutes at room temperature. For PrimeFlow experiments target probes were designed to interrogate expression of Gzma, Gzmb, Gzmf and a control probe to actin-beta. 5×10⁶ cells were prepared for all PrimeFlow samples according to the manufacturer's instructions (ThermoFisher). All flow cytometry data were analyzed using FlowJo software (Tree Star). Single color controls were used in the compensation of all flow cytometry experiments.

### Generation of Murine CD19 CAR T Cells

Granzyme over-expressing CAR constructs were designed using the pMSCV-Flag-mCD19-CD28-ZI2-tEGFR gamma-retroviral transfer plasmid (Zhuo et al., eLife 2016; 5 doi 10.7554/eLife.22429). Plasmids were synthesized by Vectorbuilder and were designated the following vector IDs: A CAR (VB210803-1134eea), B CAR (VB210803-1136cmd), and F CAR (VB210803-1139qst). Granzyme genes were inserted in place of the tEGFR sequence, directly following the CAR transcript and linked to CAR expression by a P2a linker. Retroviral vectors encoding each CAR were produced by transient transfection of the Platinum-E cell line, which stably express gag, pol, and ecotropic env genes, using Lipofectamine 3000 (Life Technologies) with plasmids encoding the CAR constructs. Viral supernatant was harvested 48 hours after transfection of Platinum-E cells and frozen in 1 mL aliquots. T cells were enriched from C57BL/6 splenocytes using a negative-selection magnetic T cell enrichment kit (StemCell). T cells were activated using 25 μL anti-CD3/CD28 beads (Life Technologies) per 1×10⁶ T cells in complete media and beads were removed 3 days after transduction. T cells were cultured at a starting concentration of 1×10⁶ cells/mL with IL-2 (40 IU/ml) (produced in house) and IL-7 (10 ng/ml) (peprotech). 2 mL of activated T cells were transduced per well of a 6 well plate, in retronectin-coated 6-well plates (Takara) with 2-3 mL of viral supernatant and on days 2 and 3 post-T cell purification. Transduction efficiency was evaluated by flow cytometry on day 5. T cell numbers were adjusted based on transduction efficiency for downstream use.

### Syngeneic in Vivo Studies

B6 mice were injected with 1×10⁶ E2a cells on day zero of in vivo studies. Zero to three days later, mice were lymphodepleted with a sub-lethal dose of total body irradiation (500 cGy using 137Cs source). Two days following irradiation mice were treated with 1×105 anti-CD19 CAR T cells (the number of CAR+ T cells was equal across cohorts adjusting for transduction efficiency). Analysis was performed at various time points as described in each experiment. CAR T cells were detected by flow cytometry on blood draws or bone marrow (flow cytometry panel described in Table 4). Data points in figures from blood and bone marrow samples represent individual mice. Submandibular blood was drawn from mice and treated with red blood cell lysis buffer before flow cytometric analyses. Bone marrow was harvested from femurs, processed into single cell suspensions, and RBCs were lysed using ACK lysis buffer. Cells were stained with antibodies and analyzed by flow cytometry.

TABLE 3
Flow cytometry panel for PrimeFlow experiments
using a 5 laser Cytek Aurora.

| | | | | | Catalogue |
|---|---|---|---|---|---|
| Antigen | Clone | Fluor | Channel | Company | # |
| Fixable | | “Blue” | UV6 | Thermo | L23105 |
| viability dye | | | | Fisher | |
| CD45 | 30-F11 | Pac | V3 | Biolegend | 103125 |
| | | Blue | | | |
| CD3 | 17A2 | BV785 | V15 | Biolegend | 100232 |
| CD8b | YTS156.7.7 | BV711 | V13 | Biolegend | 126633 |
| CD137 | 17B5 | PE/Cy7 | YG9 | Thermo | 50-112- |
| | | | | Fisher | 3331 |
| PD-1 | 29F.1A12 | BV510 | V7 | Biolegend | 135241 |
| TIM3 | B8.2C12 | BV421 | V1 | Biolegend | 134019 |
| Slamf6 | 13G3 | BV605 | V10 | BD | 745250 |
| GzmF probe | | AF647 | R2 | Thermo | Custom* |
| | | | | Fisher | |
| GzmB probe | | AF568 | YG3 | Thermo | Custom |
| | | | | Fisher | |
| GzmA probe | | AF488 | B2 | Thermo | Custom |
| | | | | Fisher | |
| Actin-beta | | AF750 | R7 | Thermo | Custom |
| (Control probe) | | | | Fisher | |

*ThermoFisher designed PrimeFlow probes by determining unique regions of the granzyme genes to target.

TABLE 4
Flow cytometry panel for in vitro CAR cytotoxicity
experiments using a 5 laser Cytek

| | | | | | Catalogue |
|---|---|---|---|---|---|
| Antigen | Clone | Fluor | Channel | Company | # |
| Fixable | | “Blue” | UV6 | Thermo | L23105 |
| viability dye | | | | Fisher | |
| Flag Tag | M2 | APC | R1 | Abcam | AD0059F |
| CD8 | 53-6.7 | PEcy7 | YG9 | Invitrogen | 53-6.7 |
| CD4 | RM4-5 | Pac blue | V3 | Biolegend | 100531 |
| GFP | | GFP | B2 | | |
| Annexin V | | APC/Fire- | R7 | Biolegend | 640953 |
| | | 750 | | | |
| CD69 | H1.2F3 | BV650 | V11 | Biolegend | 104541 |
| Calreticulin | D3E6 | PE | YG1 | Cell | 19780S |
| | | | | Signaling | |
| | | | | Technology | |

TABLE 5
Flow cytometry panel for CAR transduction
efficiency using a 5 laser Cytek Aurora.

| Antigen | Clone | Fluor | Channel | Company | Catalogue # |
|---|---|---|---|---|---|
| Fixable | | “Blue” | UV6 | Thermo | L23105 |
| viability dye | | | | Fisher | |
| Flag Tag | M2 | APC | R1 | Abcam | AD0059F |
| CD8 | 53-6.7 | PEcy7 | YG9 | Invitrogen | 53-6.7 |
| CD4 | RM4-5 | Pac blue | V3 | Biolegend | 100531 |
| CD69 | H1.2F3 | BV650 | V11 | Biolegend | 104541 |

### Discussion for Examples 1-8

The results presented in the Examples show that granzyme expression is highly regulated: specifically, Gzmf is one of the most upregulated genes in CD8 TIL relative to CD8 T cells outside the TME. It was demonstrated that TCR activation influences expression of granzymes differently and granzymes have distinct expression patterns. The results imply a dynamic system in which T cells alter their expression of different granzymes in response to the specific stimulus they receive. Gzma, Gzmb and Gzmk expression was upregulated in response to TCR stimulation, but Gzmb expression is more rapidly and highly upregulated. Expression of other granzymes tested did not change in response to TCR stimulation alone, but likely require other stimuli for their expression to be altered. This positions granzymes as useful markers of various T cell activation states and may make their expression a critical metric in distinguishing functional and ineffectual T cell responses.

Others have found that T cells taken from progressing tumors expressed significantly more Gzmf than T cells taken from regressing tumors, suggesting that Gzmf may be a marker of a suboptimal T cell response (Woolaver et al. Journal for ImmunoTherapy of Cancer 2021; 9 (1): e001615). It was determined that Gzmf is expressed specifically in exhausted CD8 TIL. The increase in expression of a cytotoxic mediator specifically during the process of exhaustion may provide insight into T cell dysfunction and could position Gzmf as a useful marker of hypofunctional T cells. Furthermore, Gzmf expression in exhausted CD8 T cells may implicate Gzmf as having an active role in T cell dysfunction.

Most studies of granzymes focus on Gzma or Gzmb. However, Gzmk, another often overlooked granzyme, has been implicated by multiple groups as a marker of unique differentiation pathways in TIL (Tiberti et al., Nature Communications 2022; 13 (1): 6752; Zheng et al. Science (New York, NY) 2021; 374 (6574)). The TCM treatment reduced Gzmk expression in response to TCR activation in T cells.

Through the overexpression of Gzma, Gzmb and Gzmf in CAR T cells it was determined that Gzmf increases the cytotoxic activity of T cells against target tumor cells; however, Gzmf overexpression induced increased cell death in the CAR T cells producing it. Furthermore, Gzmf induced an alternative cell death pathway that results in increased membrane permeabilization in dying cells, confirming what other groups have shown using recombinant granzyme F (Shi et al. Cell Death and Differentiation 2009; 16 (12): 1694-706). A CARs and B CARs induced significantly more apoptotic cell death than F CARs and A CARs had less self-directed cytotoxicity than F CARs. In vivo, overexpression of Gzma and Gzmf impaired CAR T cell control of target cell growth, to the same levels as subjects treated without CAR T adoptive transfer, but that Gzmb had no significant impact on controlling CD19-expressing cell expansion relative to Plain CARs. Additionally, there was a reduction in CD69 surface expression on granzyme overexpressing CAR T cells relative to Plain CARs, suggesting that activated T cells are specifically eliminated by the granzyme molecules they produce, resulting in reduced CD69 expression in the population overall. There are mechanisms that protect T cells from their own granzyme-mediated cytotoxic activities, such as serpins (Kaiserman D, Cell Death & Differentiation 2010; 17 (4): 586-95; Sun et al. The Journal of Biological Chemistry 1996; 271 (44): 27802-9; Hirst et al. Journal of Immunology (Baltimore, Md: 1950) 2003; 170 (2): 805-15; Masson, Molecular immunology 1988; 25 (12): 1283-9). However, the data presented herein shows that granzyme A and F are not as well protected against as granzyme B. Better protection against granzyme B expression in T cells is likely evolutionarily advantageous, as granzyme B is such a highly expressed granzyme. The lack of protection against the usually lowly expressed Gzmf may be less important and may even be beneficial to an organism if T cells need to self-eliminate.

Together, these findings imply that Gzmf-expressing effector T cells could be a short-lived population that possess improved cytotoxic capacities but are susceptible to the same mechanisms of cytotoxicity they use to eliminate target cells. Without wishing to be bound by theory, it is hypothesized that Gzmf is a marker of a cytotoxic but terminally exhausted T cell subset that will be short-lived. Self-targeted granzyme-mediated destruction of exhausted T cells could expand the paradigm to define the final steps in the pathway of terminal T cell exhaustion, by providing a mechanism by which exhausted T cells are terminated.

What is claimed:

1. A modified CAR cell comprising a chimeric antigen receptor (CAR) and an exogenous polynucleotide encoding at least one granzyme.

2. A modified CAR cell produced by introducing into the CAR cell an exogenous polynucleotide encoding at least one granzyme.

3. The modified CAR cell of claim 1 or 2, wherein the granzyme is granzyme F.

4. The modified CAR cell of any one of claims 1-3, wherein the exogenous polynucleotide comprises:
i. a MSCV promoter,
ii. a chimeric antigen receptor (CAR),
iii. at least one linker domain (L), and
iv. a granzyme gene (Gzm).

5. The modified CAR cell of any one of claims 1-4, wherein the modified CAR cell is an immune cell.

6. The modified CAR cell of claim 5, wherein the immune cell is a T-cell, a hematopoietic progenitor cell, a peripheral blood (PB) derived T-cell or an umbilical cord blood (UCB) derived T-cell.

7. The modified CAR cell of claim 5, wherein the immune cell is a CD8+ T cell.

8. The modified CAR cell of anyone of claims 1-7, wherein the granzyme gene encodes granzyme A, granzyme B, or granzyme F.

9. A method of improving a cell therapy, comprising contacting a target cell with the modified CAR cell of any one of claims 1-8, wherein the amount of granzyme produced by the modified CAR cell is increased compared to an unmodified CAR cell.

10. The method of claim 9, wherein the increased granzyme production results in an increase in cell death in the target cell as compared to contacting the target cell with an unmodified CAR cell.

11. The method of claim 9, wherein the increased granzyme production results in an altered ratio of granzymes produced by the modified CAR cell compared to an unmodified CAR cell.

12. The method of claim 9, wherein the increased granzyme production results in increased cytotoxic capacity of the modified CAR cell compared to an unmodified CAR cell.

13. The method of claim 9, wherein the modified CAR cell induces a different form of cell death in the target cell compared to an unmodified CAR cell.

14. The method of claim 13, wherein the form of cell death of the target cell is caspase-independent and/or results in rupture of the target cell membrane.

15. The method of claim 13, wherein the form of cell death of the target cell is non-apoptotic.

16. The method of any one of claims 9-15, wherein the target cell is a cancer cells

17. The method of any one of claims 9-16, wherein the cell therapy is a method of treating cancer.
